(19) United States
(12) Patent Application Publication
(10) Pub. No.: US 2023/0407288 A1
(43) Pub. Date: Dec. 21, 2023

(54) COMPOSITIONS AND METHODS FOR NUCLEIC ACID ISOLATION

(71) Applicant: LIFE TECHNOLOGIES AS, Oslo (NO)

(72) Inventors: Laure JOBERT, Hosle (NO); Marie BOSNES, Oslo (NO); Hannah Kim LINDSTROM, Oslo (US)

(21) Appl. No.: 18/252,283

(22) PCT Filed: Nov. 10, 2021
(86) PCT No.: PCT/EP2021/081305

§ 371 Date: May 9, 2023

(30) Foreign Application Priority Data
Nov. 10, 2020 (GB) 2017751.5

(51) Int. Cl. C12N 15/10; C12P 19/34

(52) U.S. Cl. CPC C12N 15/1013; C12P 19/34

(57) ABSTRACT

A method of separating RNA from a sample, comprising providing: a sample comprising RNA, a binding solution comprising an oligoethylene glycol and a salt, and a solid support having a hydrophilic surface. The method further comprises contacting the sample with the binding solution and solid support, under conditions that allow binding of the RNA in the sample to the surface of the solid support, thereby providing a solid support with bound RNA in contact with residual solution; and separating the solid support with bound RNA from the residual solution. During the binding of the RNA to the surface of the solid support, oligoethylene glycol is present at a concentration of at least about 35% v/v and the salt is present at a concentration of between about 1 M and about 2 M. Methods for producing purified RNA are also provided, comprising in vitro transcription to produce an RNA molecule with a first magnetic bead and purification with a second magnetic bead. Also provided are kits and uses.

This disclosure relates to a method of separating RNA from a sample, which may be used to purify and concentrate the RNA. The disclosure also relates to kits that may be used in such methods, as well as use of a binding solution to separate RNA from a sample solution to a solid support.

### BACKGROUND

Recent developments in mRNA-based drug technologies are showing great promise for vaccine generation, protein replacement and cancer therapy. Those technologies include the generation of specific mRNAs by in vitro transcription from a DNA template and the subsequent delivery of the mRNA into the patient. These new ground-breaking technologies demand powerful and reliable tools for the purification of the newly transcribed RNA and its subsequent up-concentration. The current gold standard RNA purification method is high-performance liquid chromatography (HPLC). However, this technique is very expensive (5-30 million US$ for 10 grams) and represents a high portion of the production cost of an mRNA drug.

Solid supports, such as Oligo(dT) beads, can provide a scalable and automation-friendly tool for the purification of poly(A)+RNA. The final elution step may, however, result in dilution of the RNA sample, which then requires an additional concentration step, adding to the time and cost of the procedure.

There is therefore a need for further effective methods of RNA purification. In particular, it would be beneficial to develop methods of RNA purification that are efficient and reduce the production cost of RNA, both for research purposes and for RNA therapeutics.

### BRIEF SUMMARY OF THE DISCLOSURE

The present invention provides efficient methods for the purification and concentration of RNA, which may be useful when seeking to isolate RNA that has been synthesized using standard procedures, such as in vitro transcription reaction. The methods may also be useful for the isolation of RNA from other samples, such as blood or serum.

In an aspect, the invention provides a method of separating RNA from a sample. The method comprises providing a sample comprising RNA, a binding solution comprising an oligoethylene glycol and a salt, and a solid support having a hydrophilic surface. The oligoethylene glycol comprises from about 2 to about 70 ethylene glycol units in linear arrangement. The method further comprises contacting the sample with the binding solution and a solid support, under conditions that allow binding of the RNA in the sample to the surface of the solid support, thereby providing a solid support with bound RNA in contact with residual solution; and separating the solid support with bound RNA from the residual solution. During the binding of the RNA in the sample to the surface of the solid support, the oligoethylene glycol is present at a concentration of at least about 35% v/v and the salt is present at a concentration of between about 1 M and about 2 M.

A second aspect provides a method for producing a purified ribonucleic acid (RNA) molecule. The method comprises fixing a first magnetic bead in place by a magnetic field, wherein an in vitro transcription (IVT) template is linked to the first magnetic bead. The first magnetic bead is contacted with a reagent mixture suitable for IVT of the template under condition in which IVT occurs, thereby producing an RNA molecule. The RNA molecule is then separated from the first magnetic bead, thereby producing the purified RNA molecule. The purified RNA molecule is contacted with a second magnetic bead under conditions that allows for the purified RNA molecule to remain associated with the second magnetic bead during washing. The second magnetic bead is then washed while the second magnet bead is fixed in place by a magnetic field. After washing, the purified RNA molecule released from association with the second magnetic bead, thereby producing a highly purified RNA molecule. The steps outlined above for the second aspect may be repeated at least once, wherein the second magnetic bead of the (or each) immediately previous cycle reused as the second magnetic bead in the following cycle, In addition, the first magnetic bead of the (or each) immediately previous cycle may also be reused as the first magnetic bead in the following cycle.

A third aspect provides a kit. The kit comprises a binding solution comprising aqueous oligoethylene glycol and a salt, wherein the oligoethylene glycol comprises from about 2 to about 70 ethylene glycol units in linear arrangement, and wherein the oligoethylene glycol is present at a concentration of at least about 35% v/v and the salt is present at a concentration of between about 1 M and about 2 M. The kit also comprises a solid support having a hydrophilic surface.

In a fourth aspect, the invention provides use of a binding solution to separate RNA from a sample solution to a solid support. The binding solution comprises an oligoethylene glycol at a concentration of at least about 45% v/v and a salt present at a concentration of between about 1.2 M and about 2.5 M. The oligoethylene glycol comprises from about 2 to about 70 ethylene glycol units in linear arrangement such as, for example, tetraethylene glycol.

In a fifth aspect, the invention provides use of magnetic beads to separate RNA from a sample solution. The magnetic beads comprise a hydrophilic surface and a saturation mass magnetization of about 30 emu/g to about 90 emu/g. The magnetic beads are reusable, such that the use comprises 2 or more cycles of separating RNA from a sample solution, each cycle comprising contacting the magnetic beads with a sample in the presence of a binding solution, separating the solid support with bound RNA from the residual solution, and contacting the separated solid support with an elution buffer.

### BRIEF DESCRIPTION OF THE DRAWINGS

Embodiments of the invention are further described hereinafter with reference to the accompanying drawings, in which:

FIG. 1 illustrates a workflow for synthesis of mRNA, followed by mRNA Oligo(dT) mRNA purification. In the first step, the mRNA is synthesised from a solid phase comprising a DNA template. The solid phase with a DNA template is then removed. In a second step, the mRNA is purified with a solid phase comprising an Oligo(dT) moiety, to capture polyadenylated mRNA. The purified mRNA is then eluted from the solid phase, with the solid phase then removed.

FIG. 2 provides an outline of an exemplary method for purifying RNA (e.g. in vitro transcribed RNA) from a sample by generic capture on a hydrophilic solid phase (such as e.g. a carboxylic acid coated bead). The method comprises binding the RNA to a solid phase in the presence of a binding buffer, at least one stage of washing to remove the (unbound) non-RNA components, then eluting the purified RNA.

FIG. 3 provides a further outline of an exemplary method for separating RNA from a sample.

FIG. 4 provides brightfield imaging of Dynabeads™ MyOne™ Carboxylic Acid beads. Dynabeads™ MyOne™ Carboxylic Acid beads were suspended in 0.5 M NaCl, 5% PEG 8000 (A) or 0.5 M NaCl, 10% PEG 8000 (B). Images were acquired on a light microscope with the 20× objective.

FIG. 5 illustrates the results obtained for RNA precipitation on MyOne™ Carboxylic Acid beads with the precipitating agent PEG 8000. Two μg of RNA markers were precipitated in either 0.5 M NaCl, 5% PEG 8000 or 0.5 M NaCl, 10% PEG 8000. The RNA concentration (A), RNA recovery rate (B), A₂₆₀/A₂₃₀ ratio (C) and A₂₆₀/A₂₃₀ ratio (D) were measured before (black bars) and after (white bars) RNA precipitation.

FIG. 6 indicates the results for quantitative and qualitative analysis of the crude and purified in vitro transcription (IVTS) RNA fractions. The amount of RNA was measured in the eluate fractions (E1-5) after purification on Dynabeads™ Oligo(dT)₂₅, the supernatant (SN) fraction and the crude IVTS RNA (A). The same samples were analyzed by electrophoresis on a 1.2% agarose gel (B).

FIG. 7 illustrates the results for precipitation of crude and purified IVTS RNAs on MyOne™ Carboxylic Acid beads. Varying amounts of RNA (2, 20, 30 or 40 μg) were precipitated on 800 μg of Dynabeads™ MyOne™ Carboxylic Acid beads. The RNA concentration (A), RNA recovery rate (B), A₂₆₀/A₂₃₀ ratio (C) and A₂₆₀/A₂₃₀ ratio (D) were measured before (white bars) and after (black bars) RNA precipitation. The precipitated RNAs were analyzed by electrophoresis on a 1.2% agarose gel (E).

FIG. 8 provides quantitative and qualitative analysis of the crude (white bars) and purified (black bars) IVTS RNA fractions after calibration of the DNA template load. The amount of RNA was measured for the different DNA template immobilization conditions before and after purification on Dynabeads™ Oligo(dT)₂₅ (A). The same samples were analyzed by electrophoresis on a 1.2% agarose gel (B).

FIG. 9 illustrates the results for precipitation of crude and purified IVTS RNAs on MyOne™ Carboxylic Acid beads using different DNA template loads for immobilization on Streptavidin beads. Various types of RNA (IVTS 1, RNA generated using beads loaded with 4 μg of DNA; IVTS 2, RNA generated using beads loaded with 4 μg of DNA and washed with stringent wash buffer; IVTS 3, RNA generated using beads loaded with 2 μg of DNA and washed with stringent wash buffer) were precipitated on Dynabeads™ MyOne™ Carboxylic Acid beads. The RNA concentration (A), RNA recovery rate (B), A₂₆₀/A₂₃₀ ratio (C) and A₂₆₀/A₂₃₀ ratio (D) were measured before (white bars) and after (black bars) RNA precipitation. The precipitated RNAs were analyzed by electrophoresis on a 1.2% agarose gel (E).

FIG. 10 provides quantification of the DNA template contamination. The crude and purified IVTS RNA samples were diluted 1:10000 and subjected to DNA quantification by qPCR. The DNA concentration (A) and the approximate copy number (B) before (white bars) and after (black bars) RNA precipitation were calculated.

FIG. 11 provides quantification of the protein contamination. The crude and purified IVTS RNA samples were analyzed for protein content on the Qubit 3 fluorometer. The protein concentration (A) and the protein amount (B) were calculated before (white bars) and after (black bars) RNA precipitation.

FIG. 12 shows the effect of the order of reagent addition on mRNA yield during generic capture clean-up of in vitro transcribed mRNA.

FIG. 13 shows the effect of EtOH drying of solid phase with precipitated RNA on handling and yield of mRNA, during generic capture clean-up of in vitro transcribed mRNA.

FIG. 14 shows a comparison of mRNA capture using different carboxylic acid activated beads.

FIG. 15 provides a schematic of an mRNA production and purification workflow including reuse loops of templated and purification beads. A biotinylated template may be generated by PCR from a plasmid or synthetic DNA fragment and immobilized on Streptavidin magnetic beads. No extra purification step is needed, since purification is achieved through immobilization of the template to Streptavidin beads. Following in vitro transcription the bead-template complex is removed by applying a magnet and can be reused multiple times in subsequent IVT reactions by adding fresh reagents. Collected mRNA is then purified on carboxylic acid functionalized magnetic beads to remove T7-polymerase, remaining NTPs and other components from the IVT reagents. Beads can be reused multiple times in subsequent rounds of mRNA purification.

FIG. 16 illustrates results obtained with a reuse loop workflow. A. illustrates an exemplary recovery rate for repeated capture of mRNA with carboxylic acid functionalized beads for two different types of bead. The black bars show results where the same MyOne™ Carboxylic Acid (MyOne COOH) beads are used for up to six cycles of mRNA capture. The white bars show the results for CA-4 beads, which have a higher level of magnetization than the MyOne™ COOH beads. 300 μg MyOne™ Carboxylic Acid and CA-4 beads were used and reused in six consecutive rounds of mRNA purification. 150 μg mRNA was added to the purification reaction in each round up to a binding volume of 300 μL, following the generic capture protocol disclosed in Example 13. B., C. and D. illustrate the RNA integrity after purification rounds 1 (B.), 3 (C.) and 5 (D.) for MyOne™ COOH beads and CA-4 beads. IVT crude dilution 1.5 μg/μL target RNA was used as control. 300 μg MyOne™ COOH and CA-4 beads were used and reused in 6 consecutive rounds of generic capture. 150 μg mRNA was added to the purification reaction in each round up to a binding volume of 300 μL. The CA-4 beads maintain high performance through at least 5 rounds of mRNA purification, whereas MyOne™ COOH performance starts to decline after the 3rd use.

FIG. 17 illustrates the mRNA recovery rate when purification is performed at pH 7 to 9. RNA quantification: Qubit fluorometer, RNA BR assay. 300 μg MyOne™ COOH beads were used to purify 150 μg mRNA in a 300 μL binding reaction. Two different lots of equal RNA binding buffer (RBB) formulations were tested across the pH range of 7.0-9.0

FIG. 18 provides an illustration of how pH can affect the integrity of the recovered RNA, as assessed using a bioanalyzer high resolution capillary electrophoresis, RNA 6000 Nano assay. 300 μg MyOne™ COOH beads were used to purify 150 μg mRNA in a 300 μL binding reaction. Two different lots of equal binding buffer formulations were tested across the pH range of 7.0-9.0. FIG. 18(A) provides the gel image, where the leftmost lane comprises markers, then Lane 1: IVT crude mRNA, lane 2: Lot 1, pH 7.0; lane 3: Lot 1, pH 7.5; lane 4: Lot 1, pH 8.0; lane 5: Lot 1, pH 8.5; lane 6: Lot 1, pH 9.02; lane 7: Lot 2, pH 7.0; lane 8: Lot 2, pH 7.5; lane 9: Lot 2, pH 8.0; lane 10: Lot 2, pH 8.5; lane 11: Lot 2, pH 9.0. FIG. 18(B) illustrates a comparison of the electropherograms for crude RNA compared to RNA purified at the indicated pH values.

FIG. 19 provides an indication of RNA purity by reference to protein exclusion at different pH values. FIG. 19(A) illustrates the RNA purity: protein to mRNA ratio at pH 7 to 9. 300 μg mRNA was purified with 600 μg Dynabeads MyOne™ COOH beads using RBB and RBB pH 7.0-9.0, in a total reaction volume of 600 μL. mRNA was eluted in elution buffer (EB) (Tris and Tris-HCl pH 7.0-8.0) with pH values according to the pH of the RBB, but not past pH 8.0. FIG. 19(B) shows protein carryover for purification with buffer at pH 7.5. 300 μg mRNA was purified with 600 μg MyOne™ COOH beads using RBB pH 7.5, in a total reaction volume of 600 μL. mRNA was eluted in EB (Tris-HCl pH 7.5). ≥99% of protein was removed from crude IVTS (left) and ≤9 ng protein per μg RNA was carried over to the eluate fraction (right).

FIG. 20 provides a schematic of part of an exemplary large scale workflow as set out in Example 14. (A) shows part 1 of the workflow, directed to template immobilization on Streptavidin (StA) beads; (B) shows part 2 of the workflow, directed to solid-phase in vitro transcription (IVT) including a StA bead reuse loop; (C) shows part 3 of the workflow is directed to generic capture and mRNA purification including a COOH bead reuse loop.

FIG. 21 provides an illustration of a scale up of mRNA production. (A) indicates the amounts of reagents used and yield of purified mRNA at 1×, 10× and 3000× scale. (B) shows the total yield for direct scale up of in vitro transcription volumes from 1× to 10×. (C) illustrates the increase in mRNA transcription yield for the period of time from 30 min to 2 hours. Scale up in small to medium scale was done by direct increase in all reagent volumes, from 100 μL to 300 mL in IVT, showing a dynamic increase in mRNA yield. Scale up to 1 L volumes of the complete workflow, including template immobilization, in vitro transcription and generic capture purification. 1 L scale IVT reaction in 1 L Syrris reactor. The temperature of the reaction was controlled with an external water bath that circulated through the jacket of the reactor, with temperature controls guided by thermometer inside the reactor and water bath. The IVT reaction conducted with 10 g Streptavidin coated beads per 10 mg template was allowed to proceed for 2 hours.

### DETAILED DESCRIPTION

The abbreviations used herein have their conventional meaning within the chemical and biological arts.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

For the avoidance of doubt, it is hereby stated that the information disclosed earlier in this specification under the heading “Background” is relevant to the invention and is to be read as part of the disclosure of the invention.

All publications, patent applications, patents, and other references mentioned herein are incorporated by reference in their entirety. In case of conflict, the present specification, including definitions, will control.

### Definitions

The following explanations of terms and methods are provided to better describe the present disclosure and to guide those of ordinary skill in the art in the practice of the present disclosure.

The term “oligoethylene glycol” includes reference to linear chains of ethylene glycol, H(OCH₂CH₂)ₙOH, where n is at least 2 and not more than 100. For example, an oligoethylene glycol may comprise from about 2 to about 70 ethylene glycol groups. Exemplary oligoethylene glycols include triethylene glycol, tetraethylenglycol, pentaethylene glycol, hexaethylene glycol, hepataethylenglycol, nonaethylene glycol, and decaethylene glycol. A preferred oligoethylene glycol of the present disclosure is tetraethylene glycol.

The term “RNA” refers to ribonucleic acid. An RNA may be one of many different types, such as messenger RNA (mRNA), transfer RNA (tRNA), ribosomal RNA (rRNA), long noncoding RNA (lncRNA), or small noncoding RNA (sncRNA) (e.g. micro RNA (miRNA), small nucleolar RNA (snoRNA), small nuclear RNA (snRNA), small-interfering RNA (siRNA), and PIWI-interacting RNA (piRNA)). The methods disclosed herein may be used with any type of RNA. Exemplary types of RNA that may be separated from samples by the methods disclosed herein include messenger mRNA and miRNA.

The term “sample” includes reference to a sample that may (and typically will) include RNA. The sample will often be in the form of an aqueous solution. The sample may be an in vitro transcription (IVT or IVTS) reaction solution, which comprises RNA (e.g. mRNA). The sample may be a biological sample, particles or fluids. The sample may be of eukaryotic or prokaryotic origin (such as mammalian, bacterial, yeast, viral etc.). In some instances, the sample may comprise a lysate. The sample may comprise plasma, serum, blood, urine or other body fluid. In some examples, the sample may comprise sncRNA (such as miRNA), tumor RNA, or cell-free or endogenous RNA.

The term “solid support” means, unless otherwise stated, a material that is substantially insoluble in a selected solvent system, or which can be readily separated (e.g., by precipitation) from a selected solvent system in which it is soluble. Such solid supports are not limited to a specific type of support, and a large number of such solid supports are available and are known to one of ordinary skill in the art. Exemplary solid supports include, but are not limited to, solid and semi-solid matrixes, such as aerogels and hydrogels, resins, beads (including magnetic beads, such as coated magnetic beads), biochips (including thin film coated biochips), microfluidic chip, a silicon chip, multi-well plates (also referred to as microtiter plates or microplates), membranes, conducting and nonconducting metals, glass (including microscope slides) and magnetic supports. A solid support may comprise magnetic beads. Solid supports useful in the practice of the present invention have a hydrophilic surface that allows binding of RNA, e.g. by non-covalent interactions. The hydrophilic surface of the solid support may comprise a charged surface in the pH range of about 6 to about 8.

The hydrophilic surface of the solid support may comprise negatively charged groups (such as acidic groups) and/or the hydrophilic surface of the solid support may comprise positively charged groups. The hydrophilic surface of the solid support may comprise acidic groups. The hydrophilic surface of the solid support may comprise carboxyl groups. The hydrophilic surface of the solid support may comprise positively charged groups, for example one or more positively ionizable groups with a pKₐ of between about 5 and 8 (e.g. with a pKₐ of between about 6 and 7). The hydrophilic surface of the solid support may comprise polyethylene imine groups, morpholine groups, alanine groups, polyhydroxy amine groups (such as Tris, Bis-Tris, and the like). The hydrophilic surface of the solid support may comprise biological buffer covalently bound thereto; for example a biological buffer as described in U.S. Pat. No. 6,914,137 B2, column 5, line 55 to column 6, line 59, covalently bound as described in said document at column 7, lines 29 to 64, the content of which is incorporated herein by reference in its entirety.

Where substituent groups are specified by their conventional chemical formulae, written from left to right, they equally encompass the chemically identical substituents that would result from writing the structure from right to left, e.g., —CH₂O— is equivalent to —OCH₂—.

The term “alcohol” means, unless otherwise stated, a compound comprising a hydroxyl group, for example an alkane or alkene substituted by a hydroxyl group. Exemplary alcohols include C₁-C₆ alcohol; such as methanol, ethanol, propanol, isopropanol, or butanol.

The term “polyol” means, unless otherwise stated, a compound comprising multiple (e.g. 2) hydroxyl groups. Exemplary polyols include, but are not limited to, a C₂-Cao polyol, where the polyol can be an alkane or alkene substituted by at least two hydroxyl groups and optionally interrupted by 1, 2 or 3 ether linkages. Particular examples of polyols include 2-methyl-1,3-propanediol, tripropylene glycol, and butanediol.

The term “alkane” means, unless otherwise stated, a straight (i.e. unbranched) or branched chain, or combination thereof, which is fully saturated, having the number of carbon atoms designated (e.g. C₁-C₁₀ means one to ten carbons). Examples of alkanes include, but are not limited to, groups such as methane, ethane, propane, n-butane, isobutane, n-pentane, n-hexane, n-heptane, homologs and isomers thereof. The term “alkene” means, unless otherwise stated, a hydrocarbon comprising one or more carbon-carbon double bonds. Examples of alkenes include, but are not limited to, groups such as ethene, propene, butene, pentene, hexene, heptene, homologs and isomers thereof.

The terms “halo” or “halogen,” by themselves or as part of another substituent, mean, unless otherwise stated, a fluorine, chlorine, bromine, or iodine atom. The term “halide” means, unless otherwise stated, fluoride, chloride, bromide, or iodide anion, e.g. present as an anion in a salt.

The term “in vitro transcription”, “in vitro transcription system”, “in vitro transcription reaction” also referred to as IVT or IVTS herein, relates to a process wherein RNA is synthesized in a cell-free system (in vitro). Typically plasmid DNA or synthetic DNA fragments may be used as template for the generation of RNA transcripts. The promoter for controlling in vitro transcription can be any promoter for any DNA-dependent RNA polymerase such as T7, T3, and SP6 RNA polymerases. In many instances a T7 promoter may be used. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. The DNA template may be linearized with a suitable restriction enzyme, before it is transcribed in vitro. The cDNA may be obtained by reverse transcription of mRNA or chemical synthesis. Moreover, the DNA template for in vitro RNA synthesis may also be obtained by gene synthesis, e.g. by synthesizing oligonucleotides and optionally assembling oligonucleotides into longer template DNA fragments.

Methods for in vitro transcription are known in the art (Geall et al. (2013) Semin. Immunol. 25(2): 152-159; Brunelle et al. (2013) Methods Enzymol. 530: 101-14). Reagents used in said method typically include: 1) a linearized DNA template with a promoter sequence that has a high binding affinity for its respective RNA polymerase such as bacteriophage-encoded RNA polymerases; 2) ribonucleoside triphosphates (NTPs) for the four bases (adenine, cytosine, guanine and uracil); 3) optionally a cap analog (e.g. m7G(5′)ppp(5′)G (m7G)); 4) a DNA-dependent RNA polymerase capable of binding to the promoter sequence within the linearized DNA template (e.g. T7, T3 or SP6 RNA polymerase); 5) optionally a ribonuclease (RNase) inhibitor to inactivate any contaminating RNase; 6) optionally a pyrophosphatase to degrade pyrophosphate, which may inhibit transcription; 7) MgCl2, which supplies Mg2+ ions as a co-factor for the polymerase; 8) a buffer to maintain a suitable pH value, which can also contain antioxidants (e.g. DTT), amines such as betaine and/or polyamines such as spermidine at optimal concentrations.

The term “magnetic” means responds to a magnetic field. For example, magnetic beads respond to a magnetic field. Magnetic materials (such as magnetic beads) disclosed herein are preferably paramagnetic or superparamagnetic. When the magnetic material is paramagnetic, the magnetic properties are switched off when the magnetic field is removed. When the magnetic material is superparamagnetic, the magnetic material becomes saturated at relatively low magnetic fields and switching off of the magnetic properties with removal of the magnetic field is very rapid/instant. Some magnetic material, e.g. iron oxides, form superparamagnetic crystals when the size of the crystals is sufficiently small (e.g. below about 15 nm scale for iron oxides).

The term “saturation mass magnetization” represents the state a magnetic material reaches when an increase in applied external magnetic field cannot further increase the magnetization of the material. For magnetic beads used in methods of purifying RNA, the saturation mass magnetization may have an influence on the speed and effectiveness of the method. It is considered an important characteristic of the magnetic behaviour of the beads. Saturation mass magnetization is typically expressed as emu/g, where emu is short for “electromagnetic unit’. To convert emu/g to A/m, one can multiply the value M in emu/g with the density in g/cm³, then the M is changed to the value in emu/cm³ or emu/cc. Because 1 emu/cc=1000 A/m, one can get the M in A/m by multiply 1000.

### Methods

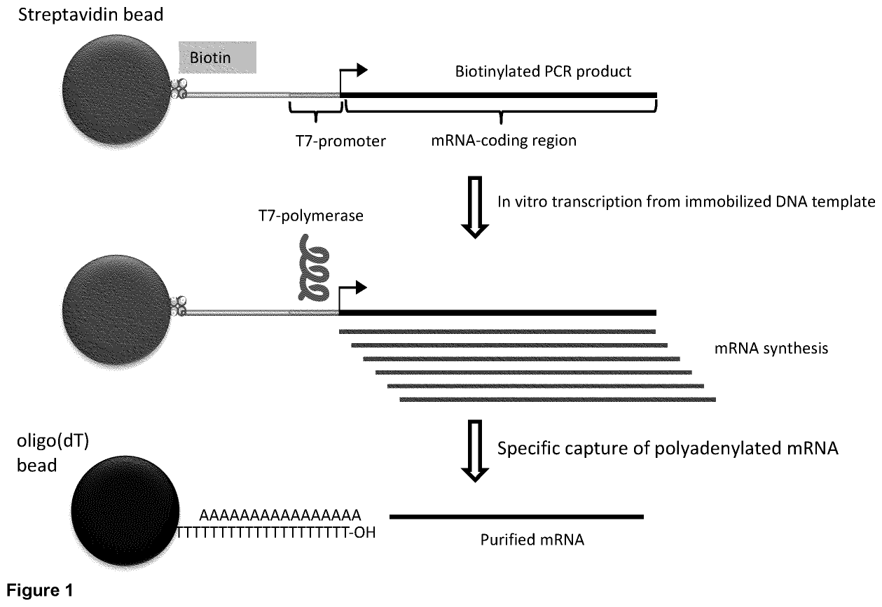

Provided herein are methods for purifying and/or up-concentrating RNA that provide advantages compared to prior art methods. In a prior art method, as illustrated in FIG. 1, mRNA can be synthesized, e.g. mRNA may be synthesized by in vitro transcription, followed by mRNA Oligo(dT) mRNA purification. In the first step, the mRNA is synthesised from a solid phase comprising a DNA template. The DNA template may comprise a T7 promoter to allow transcription of polyadenylated mRNA by a T7 RNA polymerase. In some examples, the DNA template may be biotinylated and bound to a streptavidin-coated solid phase, (such as e.g. Dynabeads™ M280 streptavidin magnetic beads). Following in vitro transcription the solid phase with a DNA template is then removed from the sample containing the transcribed mRNA (e.g., by magnetic separation). In a second step, the mRNA may be purified with a solid phase comprising an Oligo(dT) moiety, to capture poladenylated mRNA. The purified mRNA is then eluted from the solid phase, with the solid phase then removed. The mRNA obtained at the end of the second step may be obtained at a good level of purity. The mRNA may, however, be required at a higher concentration; which would require a further step of up-concentration (not illustrated).

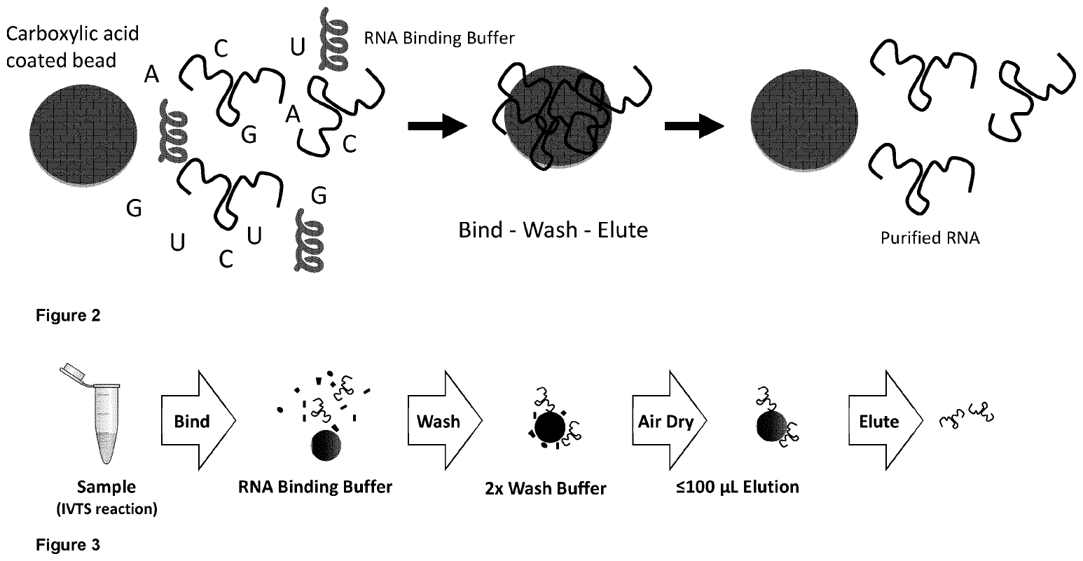

Thus, alternatively a hydrophilic solid phase may be used in the presence of a binding buffer according to various embodiments described herein to separate the mRNA to provide a higher level of concentration. The hydrophilic solid phase with bound RNA may then be subjected to a wash, elute protocol to purify and (optionally) concentrate the mRNA as illustrated in FIG. 2.

It would be advantageous to have a method that provides purified and concentrated mRNA from an in vitro transcription (or other synthetic RNA) sample with fewer steps. It would also be advantageous to provide a method that did not require a specific tag, such as polyadenylation, for separation from the sample, as this should allow purification of any RNA from the sample, e.g. endogenous RNA could be separated from samples such as blood, serum, or a lysate. The methods of the invention may provide these advantages.

In an embodiment, the invention provides a method of separating RNA from a sample. The method comprises:
a) providing a sample comprising RNA, a binding solution comprising an oligoethylene glycol and a salt, and a solid support having a hydrophilic surface;
b) contacting the sample with the binding solution and solid support, under conditions that allow binding of the RNA in the sample to the surface of the solid support, thereby providing a solid support with bound RNA in contact with residual solution; and
c) separating the solid support with bound RNA from the residual solution.
   The oligoethylene glycol comprises from about 2 to about 70 ethylene glycol units in linear arrangement. During the binding of the RNA in the sample to the surface of the solid support, the oligoethylene glycol is present at a concentration of at least about 35% v/v and the salt is present at a concentration of between about 1 M and about 2 M.

During the binding, the oligoethylene glycol may be present in a concentration of between about 35% v/v and about 60% v/v. For example, during the binding the oligoethylene glycol may be present in a concentration of between about 35% v/v and about 55% v/v, or of between about 35% v/v and about 50% v/v. During the binding the oligoethylene glycol may be present in a concentration of between about 35% v/v and about 45% v/v. During the binding the oligoethylene glycol may be present in a concentration of about 40% v/v.

The oligoethylene glycol may comprise about 2 to about 50 ethylene glycol units in a linear arrangement. For example, the oligoethylene glycol may comprise about 2 to about 30 (e.g. about 20) ethylene glycol units in a linear arrangement. The oligoethylene glycol may comprise about 2 to about 10 ethylene glycol units in a linear arrangement. The oligoethylene glycol may comprise at least 3 ethylene glycol units in a linear arrangement. The oligoethylene glycol may comprise at least 4 ethylene glycol units in a linear arrangement. The oligoethylene glycol may comprise not more than 50 (e.g. not more than 40) ethylene glycol units in a linear arrangement. The oligoethylene glycol may comprise not more than 30 (e.g. not more than 20) ethylene glycol units in a linear arrangement. The oligoethylene glycol may comprise not more than 10 ethylene glycol units in a linear arrangement. The oligoethylene glycol may be or comprise triethylene glycol, tetraethylene glycol, pentaethylene glycol, hexaethylene glycol, or heptaethylene glycol. The oligoethylene glycol may be or comprise tetraethylene glycol. The oligoethylene glycol may comprise tetraethylene glycol. The oligoethylene glycol may be tetraethylene glycol.

During the binding, the salt may be present at a concentration of between about 1 M and about 1.8 M. For example, during the binding the salt may be present at a concentration of between about 1 M and about 1.5 M. During the binding the salt may be present at a concentration of between about 1 M and about 1.25 M. During the binding the salt may be present at a concentration of about 1 M.

The salt may be selected from or comprise an alkali metal halide or an alkaline earth halide. The halide may be a fluoride, chloride or bromide. The halide may be a fluoride or chloride, e.g. a chloride. The salt may be selected from or comprise selected from or comprises sodium chloride, potassium chloride, lithium chloride, magnesium chloride, calcium chloride and barium chloride. The salt may be or comprise sodium chloride. The salt may comprise sodium chloride. The salt may be sodium chloride.

The binding solution may comprise a buffer providing a pH of from about 6 to about 9, e.g. a pH of from about 6.5 to about 8. The binding solution may comprise a buffer providing a pH of from about 7 to about 9. For example, the binding solution may comprise a buffer providing a pH of from about 7.5 to about 8.5, such as a buffer providing a pH of about 8. Preferably, the binding solution may comprise a buffer providing a pH of less than about 8, e.g. the buffer may provide a pH of from about 7 to about 8. The buffer may comprise or be selected from tris(hydroxymethyl)aminomethane (Tris), Tris/EDTA, phosphate buffered saline (PBS), citrate (e.g. sodium citrate pH 6.4), 2-(N-morpholino)ethanesulfonic acid (MES), and water. The buffer may comprise or be selected from tris(hydroxymethyl)aminomethane (Tris), Tris/EDTA, phosphate buffered saline (PBS), and water. The buffer may comprise Tris. The buffer may comprise Tris at a concentration of from about 10 mM to about 100 mM, such as from about 20 mM to about 50 mM. The buffer may comprise Tris at a concentration of from about 30 mM to about 40 mM.

A buffer may provide pH as specified due to the presence of at least one buffering agent in the buffer. The buffering agent may be present at a concentration of at least about 10 mM. The buffering agent may be present at a concentration of at least about 20 mM. The buffering agent may be present at a concentration of not more than about 150 mM. The buffering agent may be present at a concentration of not more than about 100 mM. The buffering agent may be present at a concentration of not more than about 50 mM. The buffering agent may be present at a concentration of from about 10 to about 150 mM. The buffering agent in the buffer may be present at a concentration of from about 10 to about 100 mM. The buffering agent in the buffer may be present at a concentration of from about 20 to about 100 mM. The buffering agent in the buffer may be tris(hydroxymethyl)aminomethane (Tris), Tris/EDTA, phosphate buffered saline (PBS), citrate (e.g. sodium citrate pH 6.4), 2-(N-morpholino)ethanesulfonic acid (MES), and/or water. The buffering agent in the buffer may be or comprise tris(hydroxymethyl)aminomethane (Tris), Tris/EDTA, phosphate buffered saline (PBS), and/or water. The buffering agent in the buffer may be or comprise Tris.

The sample may be a solution, for example an aqueous solution comprising RNA. The sample may comprise an in vitro transcription (IVT or IVTS) reaction solution, which comprises RNA (e.g. mRNA). The sample may be a biological sample, particles or fluids. The sample may be of eukaryotic or prokaryotic origin (such as mammalian, bacterial, yeast, viral etc.). In some instances, the sample may comprise a lysate. The sample may comprise plasma, serum, blood, urine or other body fluid. In some examples, the sample may comprise sncRNA (such as miRNA), tumor RNA, or cell-free or endogenous RNA.

In the step b) of contacting, the ratio of the volume of binding solution to volume of the sample in solution may be at least about 1 and not more than about 4. In the step b) of contacting, the ratio of the volume of binding solution to volume of the sample in solution may be at least about 1.5 and not more than about 3. In the step b) of contacting, the ratio of the volume of binding solution to volume of the sample in solution may be at least about 1.7 and not more than about 2.3. In the step b) of contacting, the ratio of the volume of binding solution to volume of the sample in solution may be about 2.

In the method, contacting the sample with the solid support and the binding solution may comprise contacting the solid support with the sample to form a mixture, then contacting the mixture with the binding solution. Contacting the solid support with the sample to form a mixture prior to addition of the binding solution may provide advantages. For example, it may avoid and/or reduce aggregation of solid support particles during step b). Avoiding and/or reducing aggregation may increase the yield of RNA separated from the sample. This may be particularly beneficial where the sample is an aqueous solution and the solid support comprises beads (such as e.g. carboxylic acid-coated beads).

The sample i) may be provided at a lower volume than the binding solution ii). For example, the sample i) may be provided at a volume of not more than about 70% of the volume of binding solution ii). The sample i) may be provided at a volume of not more than about 60% of the volume of binding solution ii). The sample i) may be provided at a volume of not more than about 50% of the volume of binding solution ii). For example, i) may be provided at a volume of not more than about 70% to not less than about 30% of the volume of binding solution ii).

The solid support having a hydrophilic surface may comprise magnetic beads. The solid support may comprise a charged surface in the pH range of about 6 to about 8. The hydrophilic surface of the solid support may comprise negatively charged groups (such as acidic groups) and/or the hydrophilic surface of the solid support may comprise positively charged groups. The surface of the solid support may comprise acidic groups, such as carboxyl groups. The solid support having a hydrophilic surface may comprise magnetic beads comprising a coating and carboxylic acid groups. The solid support having a hydrophilic surface may, for example, be or comprise exemplary magnetic beads of Table 1B.

When the solid support having a hydrophilic surface comprises magnetic beads, the magnetic beads may be paramagnetic or superparamagnetic.

When the solid support having a hydrophilic surface comprises magnetic beads, the magnetic beads may have an average diameter of from about 0.2 μm to about 10 μm. For example, the magnetic beads may have an average diameter of from about 0.2 μm to about 5 μm; e.g. the magnetic beads may have an average diameter of from about 0.3 μm to about 3 μm.

The method may further comprise: d) washing the separated solid support with bound RNA with a wash buffer and then separating the solid support with bound RNA from the residual solution (e.g. by magnetic separation). The method may further comprise repeating step d), to provide a further wash of the solid support with bound RNA. The wash buffer may be provided at an amount of at least about 100 μL (e.g. in an amount of 100-1000 μL) per mg (dry wt) of the separated solid support per wash.

The wash buffer may comprise an aqueous C₁-C₆ alcohol. The wash buffer may comprise an aqueous C₁-C₄ alcohol. The wash buffer may comprise methanol, ethanol, propanol, isopropanol, or butanol. The wash buffer may comprise ethanol. The wash buffer may comprise about 50% v/v to about 90% v/v total content of alcohol. The wash buffer may comprise about 60% v/v to about 80% v/v alcohol. The wash buffer may comprise about 50% v/v to about 90% v/v ethanol. The wash buffer may comprise about 60% v/v to about 80% v/v ethanol.

The wash buffer may comprise an aqueous C₂-C₁ polyol. The wash buffer may comprise an aqueous C₃-C₆ polyol. The wash buffer may comprise 2-methyl-1,3-propanediol, tripropylene glycol, or butanediol. The wash buffer may comprise about 40% v/v to about 70% v/v polyol. The wash buffer may comprise about 40% v/v to about 70% v/v 2-methyl-1,3-propanediol.

The wash buffer may comprise an aqueous C₁-C₆ alcohol and an aqueous C₂-C₁₀ polyol. The wash buffer may comprise an aqueous C₁-C₄ alcohol and an aqueous C₃-C₆ polyol. The wash buffer may comprise about 45% v/v to about 80% v/v total content of alcohol and polyol.

When the solid support comprises magnetic beads, the separating in step c) and/or step d) may comprise magnetic separation. Magnetic separation may comprise attracting the solid support to a magnet and then removing the residual solution (such as by decanting or pipetting). The residual solution may also be the supernatant.

The method may further comprise:
e) contacting the separated solid support with bound RNA with an elution buffer, under conditions that release the bound RNA into the elution buffer; and
f) collecting the eluate.

The elution buffer may have a pH of from about 7 to about 9. For example, the elution buffer may have a pH of from about 7.5 to about 8.5, such as a buffer having a pH of about 8. The elution buffer may be selected from or comprise aqueous Tris/EDTA, aqueous Tris, or water. The elution buffer may be or comprise aqueous Tris/EDTA.

The elution buffer may provide a pH as specified due to the presence of at least one buffering agent in the buffer. The buffering agent may be present at a concentration of at least about 5 mM or at least about 10 mM. The buffering agent may be present at a concentration of at least about 20 mM. The buffering agent may be present at a concentration of not more than about 100 mM. The buffering agent in the buffer may be present at a concentration of from about 1 mM to about 50 mM or from about 10 mM to about 100 mM. The buffering agent in the buffer may be present at a concentration of from about 20 to about 100 mM. The buffering agent in the buffer may be or comprise (Tris), Tris/EDTA, and/or water. The buffering agent in the buffer may be or comprise Tris/EDTA, optionally at a pH of about 8.

The elution buffer may be provided at a volume of less than about 100 μL per mg (dry wt) separated solid support with bound RNA. For example, the elution buffer may be provided at a volume of less than about 50 μL per mg (dry wt) separated solid support with bound RNA. The elution buffer may be provided at a volume of at least about 5 μL per mg (dry wt) separated solid support with bound RNA. The elution buffer may be provided at a volume of at least about 10 μL per mg (dry wt) separated solid support with bound RNA. The elution buffer may be provided at a volume of at least about 5 μL per mg (dry wt) separated solid support with bound RNA and not more than 100 μL per mg (dry wt) separated solid support with bound RNA. For example, the elution buffer may be provided at a volume of at least about 10 μL per mg (dry wt) separated solid support with bound RNA and not more than 50 μL per mg (dry wt) separated solid support with bound RNA; e.g. the elution buffer may be provided at a volume of at least about 15 μL per mg (dry wt) separated solid support with bound RNA and not more than 35 μL per mg (dry wt) separated solid support with bound RNA.

The volume of the elution buffer may be less than the volume of the sample comprising RNA, such that the method both purifies and concentrates the RNA from the sample. The volume of the elution buffer may be at least about 50% less than the volume of the sample comprising RNA. The volume of the elution buffer may be at least about 66% less than the volume of the sample comprising RNA. The volume of the elution buffer may be at least about 75% less than the volume of the sample comprising RNA.

The methods typically provide quantitative elution using modest volumes of the elution buffer. This provides relatively concentrated purified RNA, which has a number of advantages. This combination of purification and concentration is often useful, for example it may improve storage lifetime of the separated RNA or signal to noise during any subsequent analysis of the separated RNA or may allow for use of smaller volumes of RNA in downstream formulations (e.g. RNA vaccines).

The collecting the eluate may comprise collecting the eluate in any suitable container. The collecting the eluate may comprise any downstream processing that may be applied to purified RNA. For example, the collecting the eluate may further comprise drying the eluate, e.g. treating the RNA by lyophilisiation, and/or sterilization, and/or formulation and/or analysing the RNA.

The method may further comprise:
g) repeating steps a) to f) at least once, wherein the eluted solid support of step f) of the (or each) immediately previous cycle is used as the solid support having a hydrophilic surface in step a) of the following cycle.

Step g) may comprise repeating steps a) to f) at least 1, 2, 3, 4, 5 or 6 times. For example, step g) may comprise repeating steps a) to f) 1, 2, 3, 4, 5 or 6 times, e.g. repeating steps a) to f) 3, 4 or 5 times. Step g) may comprise repeating steps a) to f) at least 2 times. Step g) may comprise repeating steps a) to f) at least 3 times. Step g) may comprise repeating steps a) to f) at least 4 times. Step g) may comprise repeating steps a) to f) at least 5 times.

The solid support may be or comprise magnetic beads comprising a saturation mass magnetization of about 30 emu/g to about 90 emu/g. The magnetic beads may have a magnetic saturation of between about 30 and about 70 emu/g, or between about 35 and about 60 emu/g. Magnetic beads having saturation mass magnetization in this range, such as coated magnetic beads having said saturation mass magnetization, may be particularly suitable for reuse in the methods disclosed herein. For example, magnetic beads having high saturation mass magnetization may be prepared as described in U.S. Pat. No. 7,989,065 or WO 2020/018919 A1.

In the (or each) reuse cycle, the solid support may be reused without additional washing. For example, the eluted solid support of step f) of the (or each) immediately previous cycle may be reused as the solid support having a hydrophilic surface in step a) of the following cycle, without any washing of said solid support between each step g) and a).

An embodiment provides a method for producing a purified ribonucleic acid (RNA) molecule, the method comprising:
a) fixing a first magnetic bead in place by a magnetic field, wherein an in vitro transcription (IVT) template is linked to the first magnetic bead;
b) contacting the first magnetic bead of step a) with a reagent mixture suitable for IVT of the template under condition in which IVT occurs, thereby producing an RNA molecule;
c) separating the RNA molecule from the first magnetic bead, thereby producing the purified RNA molecule;
d) contacting the purified RNA molecule of step c) with a second magnetic bead under conditions that allows for the purified RNA molecule to remain associated with the second magnetic bead during washing;
e) washing of the second magnetic bead while the second magnet bead is fixed in place by a magnetic field;
f) releasing the purified RNA molecule from association with the second magnetic bead, thereby producing a highly purified RNA molecule; and
g) repeating steps a) to f) at least once, wherein the second magnetic bead of step f) of the (or each) immediately previous cycle is reused as the second magnetic bead in step d) of the following cycle, and optionally wherein the first magnetic bead of step c) of the (or each) immediately previous cycle is reused as the first magnetic bead in step a) of the following cycle.

Step g) may comprise repeating steps a) to f) at least 1, 2, 3, 4, 5 or 6 times. For example, step g) may comprise repeating steps a) to f) 1, 2, 3, 4, 5 or 6 times, e.g. repeating steps a) to f) 3, 4 or 5 times. Step g) may comprise repeating steps a) to f) at least 2 times. Step g) may comprise repeating steps a) to f) at least 3 times. Step g) may comprise repeating steps a) to f) at least 4 times. Step g) may comprise repeating steps a) to f) at least 5 times.

The first and/or second magnetic bead may comprise a saturation mass magnetization of about 30 emu/g to about 90 emu/g. The first and/or second magnetic bead may have a magnetic saturation of between about 30 and about 70 emu/g, or between about 35 and about 60 emu/g. Magnetic beads having saturation mass magnetization in this range, such as coated or functionalized magnetic beads having said saturation mass magnetization, may be particularly suitable for reuse in the methods disclosed herein.

The first and/or second magnetic beads may be paramagnetic or superparamagnetic. The first and/or second magnetic beads may be paramagnetic. The first and/or second magnetic beads may superparamagnetic.

The first and/or second magnetic beads may have an average diameter of from about 0.2 μm to about 10 μm. For example, the first and/or second magnetic beads may have an average diameter of from about 0.2 μm to about 5 μm; e.g. the first and/or second magnetic beads may have an average diameter of from about 0.3 μm to about 3 μm.

The first magnetic bead may be functionalized with streptavidin, e.g. the first magnetic bead may be functionalized with streptavidin and coated. Exemplary beads that may be used as first magnetic beads in the methods disclosed herein include those listed in the following Table 1A.

TABLE 1A

| Bead | Vendor | Bead Size | | Catalogue number |
|---|---|---|---|---|
| Dynabeads ™ M-280 ™ | Thermo Fisher | 2.8 | μm | 11205D |
| Streptavidin | Scientific | | | |
| Dynabeads ™ MyOne ™ | Thermo Fisher | 1 | μm | 65001 |
| Streptavidin | Scientific | | | |
| Bio-Adembeads | Ademtech | 200 | nm | 03123 |
| Streptavidin 200 nm, | | | | |
| 5 mg/mL | | | | |
| Bio-Masterbeads | Ademtech | 500 | nm | 03153 |
| Streptavidin 500 nm, | | | | |
| 10 mg/mL | | | | |
| Bio-Adembeads | Ademtech | 200 | nm | 03223 |
| Streptavidin plus 200 nm, | | | | |
| 5 mg/mL | | | | |
| Sera-Mag Streptavidin | Cytiva | 1 | μm | 30152105011150 |
| (High) | | | | |
| Sera-Mag SpeedBeads | Cytiva | 1 | μm | 78152104011150 |
| Neutravidin-Coated | | | | |
| Magnetic Particles | | | | |
| LodeStars 2.7 Streptavidin | Agilent | 2.7 | μm | PL6727-1003 |
| 1% (w/v) | | | | |
| LodeStars High Bind | Agilent | 2.7 | μm | PL6827-1003 |
| Streptavidin 5% (w/v) | | | | |
| MS300 Streptavidin (10%) | JSR | 3 | μm | MS300/Streptavidin, |
| | | | | 10 mL |
| MS160 Streptavidin (10%) | JSR | 1.5 | μm | MS160/Streptavidin, |
| | | | | 10 mL |
| Streptavidin Magnetic | New England | 1 | μm | S1420S |
| Beads | Biolabs | | | |
| Hydrophilic Streptavidin | New England | 2-3 | μm | S1421S |
| Magnetic Beads | Biolabs | | | |
| Streptavidin Magnetic | Merck | 0.9-1.8 | μm | SAV BE-M08/0.86 |
| Microspheres (Estapor) | | | | |
| BioMag ® Streptavidin, | Polysciences | ~1.5 | μm | 8MB4804-10 |
| Nuclease-free | | | | |
| micromer-M Streptavidin, | Micromod | 3 | μm | 08-19-303 |
| 25 mg/mL | | | | |
| Streptavidin-modified 1 μm | Vdo Biotech | 1 | μm | MS04S |
| Streptavidin-modified 3 μm | Vdo Biotech | 3 | μm | MS06S |
| Magnetic beads | Nanomicrotech | 3 | μm | |
| functionalized with | | | | |
| Streptavidin | | | | |

The second magnetic bead may be functionalized with carboxylic acid groups, e.g. the second magnetic bead may be a coated bead functionalized with carboxylic acid. Exemplary beads that may be used as second magnetic beads in the methods disclosed herein include those listed in the following Table 1B

TABLE 1B

| Bead | Vendor | Bead Size | | Catalogue number |
|---|---|---|---|---|
| Dynabeads ™ M-270 ™ | Thermo Fisher | 2.8 | μm | |
| Carboxylic Acid | Scientific | | | |
| Dynabeads ™ MyOne ™ | Thermo Fisher | 1 | μm | 65012 |
| Carboxylic Acid | Scientific | | | |
| Carboxyl-Adembeads 200 | Ademtech | 200 | nm | 02123 |
| nm, 30 mg/mL | | | | |
| Carboxyl-Masterbeads | Ademtech | 500 | nm | 02153 |
| 500 nm, 50 mg/mL | | | | |
| Sera-Mag SpeedBeads | Cytiva | 1 | μm | 45152105050350 |
| carboxylate-modified | | | | |
| (hydrofilic), 50 mg/mL | | | | |
| Sera-Mag SpeedBeads | Cytiva | 1 | μm | 65152105050350 |
| carboxylate-modified | | | | |
| (hydrofobic), 50 mg/mL | | | | |
| Beaver Beads Mag | Beaver | 1 | μm | 70104-50 |
| COOH, 10 mg/mL | Biomedical | | | |
| | Engineering Ltd | | | |
| Beaver Beads Mag | Beaver | 0.3 | μm | 70111-1 |
| COOH, 10 mg/mL | Biomedical | | | |
| | Engineering Ltd | | | |
| LodeStars 2.7 Carboxyl | Agilent | 2.7 | μm | PL6727-0003 |
| 3% (w/v) | | | | |
| MS300 Carboxyl (10%) | JSR | 3 | μm | MS300/Carboxyl, |
| | | | | 10 mL |
| MS160 Carboxyl (10%) | JSR | 1.5 | μm | MS160/Carboxyl, |
| | | | | 10 mL |
| PureProteome 1.0 μm | Millipore | 1 | μm | LSKMAG1CBX02 |
| Carboxy FlexiBind | (SigmaAldrich) | | | |
| Magnetic Bead system | | | | |
| PureProteome 2.5 μm | Millipore | 2.5 | μm | LSKMAG25CBX02 |
| Carboxy FlexiBind | (SigmaAldrich) | | | |
| Magnetic Bead System | | | | |
| 1 μm BcMag ™ Carboxy- | Bioclone | 1 | μm | FB-101 |
| terminated magnetic | | | | |
| beads | | | | |
| 1 μm BcMag ™ long-arm | Bioclone | 1 | μm | FB-105 |
| Carboxy-terminated | | | | |
| magnetic beads | | | | |
| Carboxy-Modified | Merck | 0.7-1.3 | μm | M1-070/60 |
| Uncoated Magnetic | | | | |
| Microspheres (Estapor) | | | | |
| ProMag ® 1 Series•COOH | Polysciences | 1 | μm | 25029-5 |
| Surfactant-free | | | | |
| ProMag ® 3 Series•COOH | Polysciences | 3 | μm | 86055-5 |
| Surfactant-free | | | | |
| Magnefy ™ Magnetic | Polysciences | ~1 | μm | 26410-5 |
| Microspheres | | | | |
| micromer-M COOH, 50 | Micromod | 3 | μm | 08-02-303 |
| mg/mL | | | | |
| Carboxyl Mono Mag | Ocean Nanotech | 1 | μm | XC1002 |
| Magnetic Beads, 1 μm | | | | |
| Carboxyl Mono Mag | Ocean Nanotech | 1 | μm | MC1002 |
| Magnetic Beads, 1 μm | | | | |
| Carboxyl Mono Mag | Ocean Nanotech | 3 | μm | MC3002 |
| Magnetic Beads, 3 μm | | | | |
| Carboxyl 1 μm | Vdo Biotech | 1 | μm | MS04C |
| Carboxyl 3 μm | Vdo Biotech | 3 | μm | MS06C |
| Magnetic beads | Nanomicrotech | 3 | μm | |
| functionalized with —COOH | | | | |

The conditions of contacting step d) may comprise contacting the purified RNA molecule with the second magnetic beads in the presence of a binding solution comprising an oligoethylene glycol and a salt, the oligoethylene glycol comprising from about 2 to about 70 ethylene glycol units in linear arrangement, wherein during the contacting the oligoethylene glycol is present at a concentration of at least about 35% v/v and the salt is present at a concentration of between about 1 M and about 2 M.

During the contacting, the oligoethylene glycol may be present in a concentration of between about 35% v/v and about 60% v/v. For example, during the contacting the oligoethylene glycol may be present in a concentration of between about 35% v/v and about 55% v/v, or of between about 35% v/v and about 50% v/v. During the contacting the oligoethylene glycol may be present in a concentration of between about 35% v/v and about 45% v/v. During the contacting the oligoethylene glycol may be present in a concentration of about 40% v/v.

The oligoethylene glycol may comprise about 2 to about 50 ethylene glycol units in a linear arrangement. For example, the oligoethylene glycol may comprise about 2 to about 30 (e.g. about 20) ethylene glycol units in a linear arrangement. The oligoethylene glycol may comprise about 2 to about 10 ethylene glycol units in a linear arrangement. The oligoethylene glycol may comprise at least 3 ethylene glycol units in a linear arrangement. The oligoethylene glycol may comprise at least 4 ethylene glycol units in a linear arrangement. The oligoethylene glycol may comprise not more than 50 (e.g. not more than 40) ethylene glycol units in a linear arrangement. The oligoethylene glycol may comprise not more than 30 (e.g. not more than 20) ethylene glycol units in a linear arrangement. The oligoethylene glycol may comprise not more than 10 ethylene glycol units in a linear arrangement. The oligoethylene glycol may be or comprise triethylene glycol, tetraethylene glycol, pentaethylene glycol, hexaethylene glycol, or heptaethylene glycol. The oligoethylene glycol may be or comprise tetraethylene glycol. The oligoethylene glycol may comprise tetraethylene glycol. The oligoethylene glycol may be tetraethylene glycol.

During the contacting, the salt may be present at a concentration of between about 1 M and about 1.8 M. For example, during the contacting the salt may be present at a concentration of between about 1 M and about 1.5 M. During the binding the salt may be present at a concentration of between about 1 M and about 1.25 M. During the contacting the salt may be present at a concentration of about 1 M.

The salt may be selected from or comprise an alkali metal halide or an alkaline earth halide. The halide may be a fluoride, chloride or bromide. The halide may be a fluoride or chloride, e.g. a chloride. The salt may be selected from or comprise selected from or comprises sodium chloride, potassium chloride, lithium chloride, magnesium chloride, calcium chloride and barium chloride. The salt may be or comprise sodium chloride. The salt may comprise sodium chloride. The salt may be sodium chloride.

The binding solution may comprise a buffer providing a pH of from about 6 to about 9, e.g. a pH of from about 6.5 to about 8. The binding solution may comprise a buffer providing a pH of from about 7 to about 9. For example, the binding solution may comprise a buffer providing a pH of from about 7.5 to about 8.5, such as a buffer providing a pH of about 8. Preferably, the binding solution may comprise a buffer providing a pH of less than about 8, e.g. the buffer may provide a pH of from about 7 to about 8. The buffer may comprise or be selected from tris(hydroxymethyl)aminomethane (Tris), Tris/EDTA, phosphate buffered saline (PBS), citrate (e.g. sodium citrate pH 6.4), 2-(N-morpholino)ethanesulfonic acid (MES), and water. The buffer may comprise or be selected from tris(hydroxymethyl)aminomethane (Tris), Tris/EDTA, phosphate buffered saline (PBS), and water. The buffer may comprise Tris. The buffer may comprise Tris at a concentration of from about 10 mM to about 100 mM, such as from about 20 mM to about 50 mM. The buffer may comprise Tris at a concentration of from about 30 mM to about 40 mM.

A buffer may provide pH as specified due to the presence of at least one buffering agent in the buffer. The buffering agent may be present at a concentration of at least about 10 mM. The buffering agent may be present at a concentration of at least about 20 mM. The buffering agent may be present at a concentration of not more than about 150 mM. The buffering agent may be present at a concentration of not more than about 100 mM. The buffering agent may be present at a concentration of not more than about 50 mM. The buffering agent may be present at a concentration of from about 10 to about 150 mM. The buffering agent in the buffer may be present at a concentration of from about 10 to about 100 mM. The buffering agent in the buffer may be present at a concentration of from about 20 to about 100 mM. The buffering agent in the buffer may be tris(hydroxymethyl)aminomethane (Tris), Tris/EDTA, phosphate buffered saline (PBS), citrate (e.g. sodium citrate pH 6.4), 2-(N-morpholino)ethanesulfonic acid (MES), and/or water. The buffering agent in the buffer may be or comprise tris(hydroxymethyl)aminomethane (Tris), Tris/EDTA, phosphate buffered saline (PBS), and/or water. The buffering agent in the buffer may be or comprise Tris.

Step e) washing may comprise use of a wash buffer comprising an aqueous C₁-C₆ alcohol, and/or an aqueous C₂-C₁ polyol.

The wash buffer may comprise an aqueous C₁-C₆ alcohol. The wash buffer may comprise an aqueous C₁-C₄ alcohol. The wash buffer may comprise methanol, ethanol, propanol, isopropanol, or butanol. The wash buffer may comprise ethanol. The wash buffer may comprise about 50% v/v to about 90% v/v total content of alcohol. The wash buffer may comprise about 60% v/v to about 80% v/v alcohol. The wash buffer may comprise about 50% v/v to about 90% v/v ethanol. The wash buffer may comprise about 60% v/v to about 80% v/v ethanol.

The wash buffer may comprise an aqueous C₂-C₁ polyol. The wash buffer may comprise an aqueous C₃-C₆ polyol. The wash buffer may comprise 2-methyl-1,3-propanediol, tripropylene glycol, or butanediol. The wash buffer may comprise about 40% v/v to about 70% v/v polyol. The wash buffer may comprise about 40% v/v to about 70% v/v 2-methyl-1,3-propanediol.

The wash buffer may comprise an aqueous C₁-C₆ alcohol and an aqueous C₂-C₁₀ polyol. The wash buffer may comprise an aqueous C₁-C₄ alcohol and an aqueous C₃-C₆ polyol. The wash buffer may comprise about 45% v/v to about 80% v/v total content of alcohol and polyol.

The IVT template may be synthesized de novo or produced by polymerase chain reaction (PCR). Further, one or more biotinylated primer may be used in the PCR, resulting in the formation of a biotinylated IVT template. Additionally, a biotinylated IVT template may be attached to the magnetic bead through an interaction between the biotin of the biotinylated IVT template and a group on the magnetic bead with affinity for biotin (e.g., avidin, streptavidin, etc.).

In some instances, the IVT template may comprise an open reading frame encoding a protein and a promoter (e.g. a T7 promoter) operably connected to the open reading frame.

While the second magnetic bead may associate with RNA in a number of different ways, in some instances, free carboxylic acid groups may be present on the surface of these beads.

Further, purified RNA or highly purified RNA produced as set out herein may be messenger RNA (mRNA), such as mRNA encoding one or more protein of pathogen. Further provided herein are vaccine composition comprising such mRNA or protein encoded by such mRNA.

mRNA used in such vaccine compositions may be contained in, for example, lipid nanoparticles (see, e.g., WO 2021/159130 A2, the content of which is incorporated by reference herein in its entirety).

Another embodiment provides use of a binding solution to separate RNA from a sample solution to a solid support. The binding solution comprises an oligoethylene glycol at a concentration of at least about 35% v/v and a salt present at a concentration of between about 1 M and about 2 M. The oligoethylene glycol comprises from about 2 to about 70 ethylene glycol units in linear arrangement.

In the binding solution, the oligoethylene glycol may be present in a concentration of between about 35% v/v and about 60% v/v. For example, in the binding solution the oligoethylene glycol may be present in a concentration of between about 35% v/v and about 55% v/v, or of between about 35% v/v and about 50% v/v. In the binding solution the oligoethylene glycol may be present in a concentration of between about 35% v/v and about 45% v/v. In the binding solution the oligoethylene glycol may be present in a concentration of about 40% v/v.

In the binding solution, the oligoethylene glycol may be present in a concentration of between about 50% v/v and about 80% v/v. For example, in the binding solution the oligoethylene glycol may be present in a concentration of between about 55% v/v and about 70% v/v. In the binding solution the oligoethylene glycol may be present in a concentration of or of between about 55% v/v and about 65% v/v. In the binding solution the oligoethylene glycol may be present in a concentration of about 60% v/v.

The oligoethylene glycol may comprise about 2 to about 50 ethylene glycol units in a linear arrangement. For example, the oligoethylene glycol may comprise about 2 to about 30 (e.g. about 20) ethylene glycol units in a linear arrangement. The oligoethylene glycol may comprise about 2 to about 10 ethylene glycol units in a linear arrangement. The oligoethylene glycol may comprise at least 3 ethylene glycol units in a linear arrangement. The oligoethylene glycol may comprise at least 4 ethylene glycol units in a linear arrangement. The oligoethylene glycol may comprise not more than 50 (e.g. not more than 40) ethylene glycol units in a linear arrangement. The oligoethylene glycol may comprise not more than 30 (e.g. not more than 20) ethylene glycol units in a linear arrangement. The oligoethylene glycol may comprise not more than 10 ethylene glycol units in a linear arrangement. The oligoethylene glycol may be or comprise triethylene glycol, tetraethylene glycol, pentaethylene glycol, hexaethylene glycol, or heptaethylene glycol. The oligoethylene glycol may be or comprise tetraethylene glycol. The oligoethylene glycol may comprise tetraethylene glycol. The oligoethylene glycol may be tetraethylene glycol.

In the binding solution, the salt may be present at a concentration of between about 1 M and about 1.8 M. For example, in the binding solution the salt may be present at a concentration of between about 1 M and about 1.5 M. In the binding solution the salt may be present at a concentration of between about 1 M and about 1.25 M. In the binding solution the salt may be present at a concentration of about 1 M.

In the binding solution, the salt may be present at a concentration of between about 1.2 M and about 2 M. For example, in the binding solution the salt may be present at a concentration of between about 1.3 M and about 1.8 M. In the binding solution, the salt may be present at a concentration of about 1.5 M.

The salt may be selected from or comprise an alkali metal halide or an alkaline earth halide. The halide may be a fluoride, chloride or bromide. The halide may be a fluoride or chloride, e.g. a chloride. The salt may be selected from or comprise selected from or comprises sodium chloride, potassium chloride, lithium chloride, magnesium chloride, calcium chloride and barium chloride. The salt may be or comprise sodium chloride. The salt may comprise sodium chloride. The salt may be sodium chloride.

The binding solution may comprise a buffer providing a pH of from about 6 to about 9, e.g. a pH of from about 6.5 to about 8. The binding solution may comprise a buffer providing a pH of from about 7 to about 9. For example, the binding solution may comprise a buffer providing a pH of from about 7.5 to about 8.5, such as a buffer providing a pH of about 8. Preferably, the binding solution may comprise a buffer providing a pH of less than about 8, e.g. the buffer may provide a pH of from about 7 to about 8. The buffer may comprise or be selected from tris(hydroxymethyl)aminomethane (Tris), Tris/EDTA, phosphate buffered saline (PBS), citrate (e.g. sodium citrate pH 6.4), 2-(N-morpholino)ethanesulfonic acid (MES), and water. The buffer may comprise or be selected from tris(hydroxymethyl)aminomethane (Tris), Tris/EDTA, phosphate buffered saline (PBS), and water. The buffer may comprise Tris. The buffer may comprise Tris at a concentration of from about 10 mM to about 100 mM, such as from about 20 mM to about 50 mM. The buffer may comprise Tris at a concentration of from about 30 mM to about 40 mM. The buffer may comprise Tris at a concentration of about 25 mM.

The sample solution may be an aqueous solution comprising RNA. The sample may comprise an in vitro transcription reaction solution, or a biological sample (e.g. blood, plasma, serum, urine, or a lysate). The sample may comprise an in vitro transcription reaction. The sample may comprise plasma, serum, blood, urine or other body fluid. The sample may comprise a lysate.

The sample solution may be provided at a lower volume than the binding solution. For example, the sample solution may be provided at a volume of not more than about 70% of the volume of binding solution. The sample solution may be provided at a volume of not more than about 60% of the volume of binding solution. The sample solution may be provided at a volume of not more than about 50% of the volume of binding solution. For example, the sample solution may be provided at a volume of not more than about 70% to not less than about 30% of the volume of binding solution.

The solid support may comprise magnetic beads. The solid support may comprise a hydrophilic surface. The solid support may comprise a charged surface in the pH range of about 6 to about 8. The surface of the solid support may comprise negatively charged groups (such as acidic groups) and/or the surface of the solid support may comprise positively charged groups. The surface of the solid support may comprise acidic groups, such as carboxyl groups. The solid support may comprise magnetic beads comprising a coating and carboxylic acid groups. The solid support may, for example, be or comprise exemplary magnetic beads of Table 1B.

When the solid support comprises magnetic beads, the magnetic beads may be paramagnetic or superparamagnetic.

When the solid support comprises magnetic beads, the magnetic beads may have an average diameter of from about 0.2 μm to about 10 μm. For example, the magnetic beads may have an average diameter of from about 0.2 μm to about 5 μm; e.g. the magnetic beads may have an average diameter of from about 0.3 μm to about 3 μm.

In an embodiment, the invention provides use of magnetic beads to separate RNA from a sample solution. The magnetic beads comprise a hydrophilic surface and a saturation mass magnetization of about 30 emu/g to about 90 emu/g. The magnetic beads are reusable, such that the use comprises 2 or more cycles of separating RNA from a sample solution, each cycle comprising contacting the magnetic beads with a sample in the presence of a binding solution, separating the magnetic beads with bound RNA from the residual solution, and contacting the separated magnetic beads with bound RNA with an elution buffer. The separated magnetic beads with bound RNA may be washed with a wash buffer prior to contacting the separated magnetic beads with the elution buffer.

Each of the binding solution, wash buffer and elution buffer may be as defined elsewhere herein (e.g. as defined above in the “Methods” section).

The magnetic beads may comprise a charged surface in the pH range of about 6 to about 8. The hydrophilic surface of the magnetic beads may comprise negatively charged groups (such as acidic groups) and/or the hydrophilic surface of the magnetic beads may comprise positively charged groups. The surface of the magnetic beads may comprise acidic groups, such as carboxyl groups. The magnetic beads may comprise a coating and carboxylic acid groups. The magnetic beads may, for example, be or comprise exemplary magnetic beads of Table 1B.

The magnetic beads may be paramagnetic or superparamagnetic.

The magnetic beads may have an average diameter of from about 0.2 μm to about 10 μm. For example, the magnetic beads may have an average diameter of from about 0.2 μm to about 5 μm; e.g. the magnetic beads may have an average diameter of from about 0.3 μm to about 3 μm.

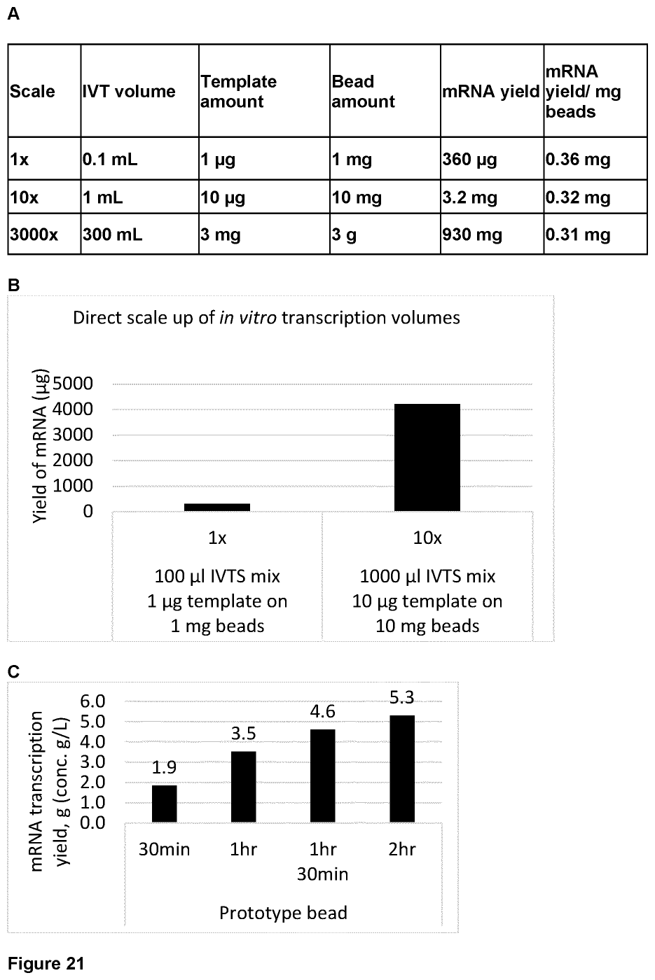

As the skilled person will appreciate, all of the methods and uses disclosed herein may be used at different scales. For example, we have demonstrated that exemplary methods provided herein scale approximately linearly from the microgram to gram scale (see, e.g., FIG. 21). Accordingly, the methods of the invention may be used to provide RNA in any desired amount.

Further, methods and uses disclosed herein may be automated or used on automated or integrated systems or devices such as those described in patent application No. PCT/US2021/054490, which is incorporated by reference herein in its entirety.

### Kits

Another embodiment provides a kit. The kit comprises a binding solution comprising aqueous oligoethylene glycol and a salt, wherein the oligoethylene glycol comprises from about 2 to about 70 ethylene glycol units in linear arrangement, and wherein the oligoethylene glycol is present at a concentration of at least about 35% v/v and the salt is present at a concentration of between about 1 M and about 2 M. The kit also comprises a solid support having a hydrophilic surface.

The binding solution may be as further defined elsewhere in the disclose, for example the binding solution may be as further defined above under the heading “Methods”.

In the binding solution, the oligoethylene glycol may be present in a concentration of between about 50% v/v and about 80% v/v. For example, in the binding solution the oligoethylene glycol may be present in a concentration of between about 55% v/v and about 70% v/v. In the binding solution the oligoethylene glycol may be present in a concentration of or of between about 55% v/v and about 65% v/v. In the binding solution the oligoethylene glycol may be present in a concentration of about 60% v/v.

In the binding solution, the salt may be present at a concentration of between about 1.2 M and about 2 M. For example, in the binding solution the salt may be present at a concentration of between about 1.3 M and about 1.8 M. In the binding solution, the salt may be present at a concentration of about 1.5 M.

The kit may further comprise a wash buffer comprising an aqueous C₁-C₆ alcohol. The wash buffer may further comprise an aqueous C₂-C₁ polyol. The wash buffer may further comprise an aqueous C₁-C₆ alcohol and an aqueous C₂-C₁ polyol. The wash buffer may comprise about 50% v/v to about 90% v/v ethanol, e.g. about 60% v/v to about 80% v/v ethanol. The wash buffer may comprise about 50% v/v to about 80% v/v 2-methyl-1,3-propanediol, e.g. about 55% v/v to about 65% v/v 2-methyl-1,3-propanediol. The wash buffer may be as may be as further defined elsewhere in the disclosure, for example the wash buffer may be as further defined above under the heading “Methods”.

The kit may further comprise an elution buffer. The elution buffer may be or comprise Tris/EDTA, Tris, and/or water. The elution buffer may comprise Tris EDTA at pH 8. The elution buffer may be as may be as further defined elsewhere in the disclosure, for example the elution buffer may be as further defined above under the heading “Methods”.

### Assays

Properties of the beads, in particular the magnetic properties of the beads used in the methods disclosed herein, may be determined in accordance with any suitable method, such as the methods disclosed in the standard ISO/TS 19807-2:2021 (en), Nanotechnologies—Magnetic nanomaterials—Part 2: Specification of characteristics and measurement methods for nanostructured magnetic beads for nucleic acid extraction, the content of which is incorporated by reference herein in its entirety. Of particular relevance are the methods disclosed at 5.2.1 to 5.2.11 of ISO/TS 19807-2:2021 (en), each of which is incorporated herein by reference.

An important property is the saturation mass magnetization. For the measurement of saturation mass magnetization, the magnetic beads shall be washed and dried in an oven. Their mass is determined by weighing. The magnetic moment of the dried beads sample is measured using a superconducting quantum interference device (SQUID) or vibrating sample magnetometry (VSM). During the measurement, the dried magnetic beads sample are susceptible to an increasing magnetic field until the value of the magnetic moment is no longer changing with field increase. At this field strength, the magnetic moment of the sample is measured. The saturation mass magnetization is calculated as the ratio of the measured magnetic moment and the mass of the magnetic beads. The result of the measurement is typically expressed in the unit A·m²/kg or emu/g.

### Further Embodiments

The disclosure and invention further include the subject matter of the following numbered clauses:
1. A method of separating RNA from a sample, the method comprising:
   a) providing
      i) a sample comprising RNA;
      ii) a binding solution comprising an oligoethylene glycol and a salt, the oligoethylene glycol comprising from about 2 to about 70 ethylene glycol units in linear arrangement; and
      iii) a solid support having a hydrophilic surface;
   b) contacting the sample with the binding solution and solid support, under conditions that allow binding of the RNA in the sample to the surface of the solid support, thereby providing a solid support with bound RNA in contact with residual solution; and
   c) separating the solid support with bound RNA from the residual solution;
   wherein, during the binding of the RNA in the sample to the surface of the solid support, the oligoethylene glycol is present at a concentration of at least about 35% v/v and the salt is present at a concentration of between about 1 M and about 2 M.
2. The method of clause 1, wherein during the binding, the oligoethylene glycol is present in a concentration of between about 35% v/v and about 50% v/v.
3. The method of clause 1 or clause 2, wherein the oligoethylene glycol comprises about 2 to about 20 ethylene glycol units in linear arrangement;
   optionally wherein the oligoethylene glycol is or comprises triethylene glycol, tetraethylene glycol, pentaethylene glycol, hexaethylene glycol, or heptaethylene glycol.
4. The method of any preceding clause, wherein the oligoethylene glycol is or comprises tetraethylene glycol.
5. The method of any preceding clause, wherein the salt is selected from or comprises an alkali metal halide, or an alkaline earth metal halide;
   optionally wherein the salt is selected from or comprises sodium chloride, potassium chloride, lithium chloride, magnesium chloride, calcium chloride and barium chloride.
6. The method of any preceding clause, wherein the salt is or comprises sodium chloride.
7. The method of any preceding clause, wherein the binding solution comprises buffer providing a pH of from about 7 to about 9;
   optionally wherein the buffer comprise Tris, Tris/EDTA, sodium citrate, PBS and water.
8. The method of clause 7, wherein the buffer comprises Tris, optionally at a concentration of from about 10 mM to about 100 mM.
9. The method of any preceding clause, wherein the sample i) comprises an in vitro transcription reaction.
10. The method of any preceding clause, wherein the sample i) is provided as a solution.
11. The method of clause 10, wherein said contacting the sample with the solid support and the binding solution comprises contacting the solid support with the sample to form a mixture, then contacting the mixture with the binding solution.
12. The method of clause 10 or clause 11, wherein the sample i) is provided at a lower volume than the binding solution ii), optionally wherein the sample i) is provided at a volume of not more than about 60% of the volume of binding solution ii).
13. The method of any preceding clause, wherein the method further comprises:
   d) washing the separated solid support with bound RNA with a wash buffer and then separating the solid support with bound RNA from the residual solution,
   the wash buffer comprising an aqueous C₁-C₆ alcohol, and/or an aqueous C₂-C₁₀ polyol.
14. The method of clause 13, wherein the wash buffer comprises about 50% v/v to about 90% v/v ethanol, optionally about 60% v/v to about 80% v/v ethanol.
15. The method of clause 13 or clause 14, further comprising repeating step d).
16. The method of any preceding clause, further comprising:
   e) contacting the separated solid support with bound RNA with an elution buffer, under conditions that release the bound RNA into the elution buffer; and
   f) collecting the eluate.
17. The method of clause 16, wherein the elution buffer is or comprises Tris/EDTA, Tris, or water;
   optionally wherein the elution buffer comprises Tris/EDTA at pH 8.
18. The method of clause 16 or clause 17, wherein the elution buffer is provided at a volume of less than 100 μL per mg (dry wt) separated solid support with bound RNA.
19. The method of any of clauses 16 to 18, wherein the volume of elution buffer is less than the volume of the sample comprising RNA, such that the method both purifies and concentrates the RNA from the sample.
20. The method of clause 19, wherein the volume of the elution buffer is at least about 50% less than the volume of the sample comprising RNA; optionally wherein the volume of the elution buffer is at least about 66% less than the volume of the sample comprising RNA; further optionally wherein the volume of the elution buffer is at least about 75% less than the volume of the sample comprising RNA.
21. A kit comprising:
   a) a binding solution comprising aqueous oligoethylene glycol and a salt, wherein the oligoethylene glycol comprises from about 2 to about 70 ethylene glycol units in linear arrangement, and wherein the oligoethylene glycol is present at a concentration of at least about 35% v/v and the salt is present at a concentration of between about 1 M and about 2 M; and
   b) a solid support having a hydrophilic surface.
22. The kit of clause 21, wherein the binding solution is as further defined in any of claims 2 to 8.
23. The kit of clause 21 or 22, further comprising a wash buffer comprising an aqueous C₁-C₆ alcohol and/or an aqueous C₂-C₁₀ polyol,
   optionally wherein the wash buffer comprises about 50% v/v to about 90% v/v ethanol,
   further optionally wherein the wash buffer comprises about 60% v/v to about 80% v/v ethanol.
24. The kit of any of clause 21 to 23, further comprising an elution buffer;
   optionally wherein the elution buffer is or comprises Tris/EDTA, Tris, or water; further optionally wherein the elution buffer comprises Tris/EDTA at pH 8.
25. Use of a binding solution to separate RNA from a sample solution to a solid support,
   wherein the binding solution comprises an oligoethylene glycol at a concentration of at least about 40% v/v and a salt present at a concentration of between about 1.2 M and about 2.5 M,
   wherein the oligoethylene glycol comprises from about 2 to about 70 ethylene glycol units in linear arrangement.
26. The use of clause 25, wherein the oligoethylene glycol comprises about 2 to about 20 ethylene glycol units in linear arrangement;
   optionally wherein the oligoethylene glycol is or comprises triethylene glycol, tetraethylene glycol, pentaethylene glycol, hexaethylene glycol, or heptaethylene glycol;
   further optionally wherein the oligoethylene glycol is or comprises tetraethylene glycol.
27. The use of clause 25 or clause 26, wherein the oligoethylene glycol is present in a concentration of between about 50% v/v and about 70% v/v.
28. The use of any of clause 25 to 27, wherein the salt is selected from or comprises an alkali metal halide, or an alkaline earth metal halide;
   optionally wherein the salt is selected from or comprises sodium chloride, potassium chloride, lithium chloride, magnesium chloride, calcium chloride and barium chloride;
   further optionally wherein the salt is or comprises sodium chloride.
29. The use of any of clauses 25 to 28, wherein the binding solution comprises buffer providing a pH of from about 7 to about 9;
   optionally wherein the buffer is selected from Tris, Tris/EDTA, PBS and water.
30. The use of clause 29, wherein the buffer comprises Tris, optionally at a concentration of from about 10 mM to about 100 mM.

A detailed description of the invention having been provided above, the following examples are given for the purpose of illustrating the invention and shall not be construed as being a limitation on the scope of the invention or claims.

### EXAMPLES

The following reagents were used in the Examples:

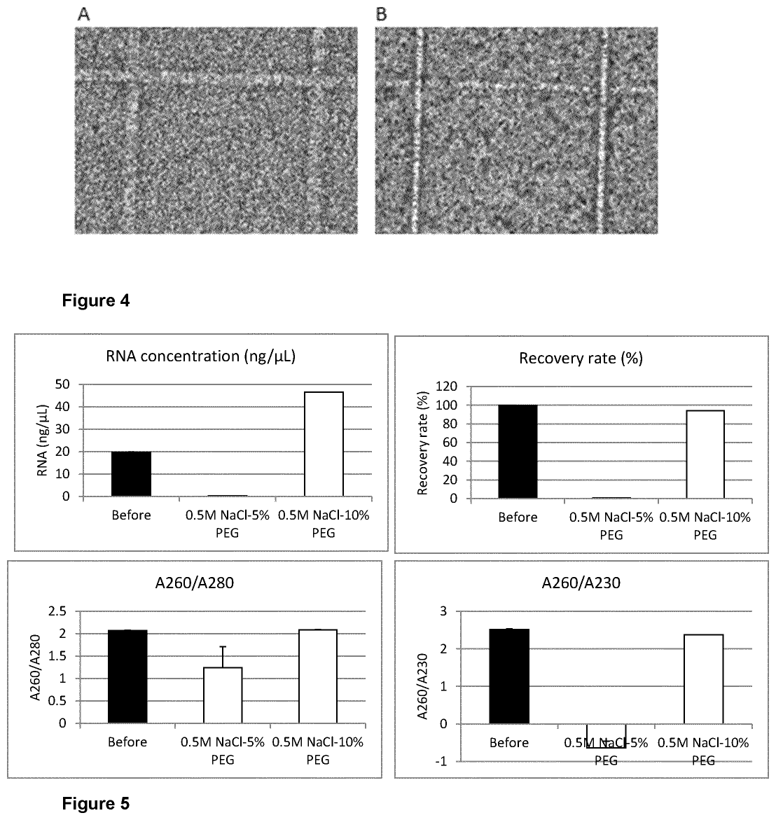

Dynabeads™ M-270™ Carboxylic Acid, Dynabeads™ MyOne™ Carboxylic Acid (also referred to as MyOne™ COOH), Dynabeads™ Oligo(dT)₂₅, and Dynabeads™ M-280™ Streptavidin (all available from Thermo Fisher Scientific) and various polystyrene beads functionalized with carboxylic acid (CA-1, CA-2, CA-3) were used as solid phase. FIG. 4 provides an image of MyOne™ COOH beads in suspension, obtained with a light microscope using the 20× objective.

The oligonucleotides used were approximately 20 bases in length and were purchased dry from BioSearch Technologies (LGC). Table 2 lists buffers and Table 3 lists other reagents. Table 4 lists equipment used in accordance with the Examples.

TABLE 2
Buffers

| Application | Name | Composition |
|---|---|---|
| RNA precipitation | 1.5X RNA Binding | 60% v/v tetraethylene glycol (TEG), 1.5M NaCl, |
| | Buffer (RBB) | 37.5 mM Tris-HCl pH 8.0 |
| | Wash Buffer (WB) | 70% v/v ethanol solution in UltraPure ™ |
| | | DNase/RNase-Free Water |
| | Elution Buffer (EB) | Tris/EDTA (TE) pH 8.0 |
| DNA immobilization | 2X StA Bind/Wash | 10 mM Tris HCl pH 7.5, 1 mM |
| on Dynabeads ™ M- | Buffer | ethylenediaminetetraacetic acid (EDTA), 2M |
| 280 Streptavidin | | NaCl |
| | Stringent Wash | 5 mM Tris HCl pH 7.5, 0.5 mM EDTA, 150 mM |
| | Buffer | NaCl, 0.1% Tween-20 |
| RNA purification on | 2X Binding Buffer | 0.2M 2-(N-morpholino)ethanesulfonic acid |
| Dynabeads ™ | | (MES) buffer pH 6.0 containing 1M LiCl and |
| Oligo(dT)₂₅ | | 20 mM EDTA |
| | Washing buffer | 0.01M MES buffer pH 6.0 containing 0.15M |
| | | LiCl and 1 mM EDTA |
| | Elution Buffer | 10 mM Tris HCl pH 7.5 |

TABLE 3
Other reagents

| Name | Vendor |
|---|---|
| Isopropanol | Sigma-Aldrich |
| Ethanol | Honeywell |
| SDS | Sigma |
| β-Mercaptoethanol | Sigma |
| Guanidinium thiocyanate | Sigma |
| N-Lauryl Sarcosine | Sigma |
| NaCl (5M) | Ambion |
| LiCl | Merck |
| MgCl₂ | Sigma |
| CuCl₂ | Riedel-de Haën |
| 0.5M EDTA pH 8.0 | Thermo Fisher Scientific |
| 1M Tris HCl pH 8.0 | Gibco |
| 1M Tris HCl buffer pH 7.5 | Thermo Fisher Scientific |
| PEG 6000 | Fluka |
| PEG 8000 | Fluka |
| PEG 10000 | Fluka |
| TEG 99% | Sigma-Aldrich |
| PCR Super Mix | Thermo Fisher Scientific |
| EcoR I | Thermo Fisher Scientific |
| MEGAscript T7 Transcription Kit | Thermo Fisher Scientific |
| DNase I RNase free | Ambion |
| RNase Cocktail Enzyme Mix | Ambion |
| E-gel 1.2% Agarose SYBR Safe | Thermo Fisher Scientific |
| RNA Millennium Marker | Ambion |
| Gel loading buffer II | Thermo Fisher Scientific |
| 1 kb Plus DNA ladder | Thermo Fisher Scientific |
| 10X BlueJuice Gel Loading Buffer | Thermo Fisher Scientific |
| 2X SYBR Green PCR Master Mix | Thermo Fisher Scientific |
| Qubit Protein Assay kits | Thermo Fisher Scientific |

TABLE 4
Equipment

| Instrument | Name | Vendor |
|---|---|---|
| Magnet | DynaMag-2 | Thermo Fisher Scientific |
| Light microscope | DM1000 LED | Leica |
| Spectrophotometer | NanoDrop One^{C} | Thermo Fisher Scientific |
| Real time PCR | 7500 System | Applied BioSystems |
| Thermo mixer | ThermoMixer C | Eppendorf |
| Electrophoresis device | E-gel PowerBase | Thermo Fisher Scientific |
| Imaging system | Chemidoc | Bio-Rad |
| Fluorometer | Qubit 3 | Thermo Fisher Scientific |
| DOE software | MODDE Pro | Umetrics |

### Example 1—RNA Precipitation

RNA may be precipitated in accordance with the following protocol, which represents an example of the workflow illustrated in FIG. 2. This protocol is suitable for the precipitation of 2-40 μg of RNA with 800 μg of Dynabeads™ MyOne™ Carboxylic Acid beads. As the skilled person would appreciate, Dynabeads™ MyOne™ Carboxylic Acid beads represent an example of a solid phase having a hydrophilic surface. The skilled person would be able to readily adapt this procedure for use with other acid functionalised hydrophilic solid phases.
1. Thoroughly suspend the beads by placing on a roller at RT for 20 min prior to use.
2. Add 80 μL of Dynabeads™ MyOne™ Carboxylic Acid beads (0.8 mg) to a 1.5 mL microtube.
3. Place the tube on the DynaMag™-2 magnet for 30 sec or until the supernatant is completely cleared. Carefully aspirate and discard the supernatant.
4. Add 200 μL of 1.5×RBB and mix using a quick pulse vortex.
5. To 100 μL of RNA sample, add 200 μL of the beads suspended in 1.5×RBB from step 4.
6. Mix the sample by inverting the tubes 3-4 times and incubate for 10 min at RT on a roller.
7. Quickly spin down and place the tube on the magnet for 30 sec.
8. Carefully aspirate and discard the supernatant.
9. Add 500 μL of WB and vortex until the beads are thoroughly suspended.
10. Place the tube on the magnet for 30 sec. Carefully aspirate and discard the supernatant.
11. Repeat steps 9 and 10 once (total of two washes).
12. Let the beads dry on the magnet at RT for 10 min.
13. Remove the magnet and add 20 μL of EB. Vortex the sample, quickly spin down and incubate at 65° C. for 5 min.
14. Vortex the beads for 10 sec and place the tube on the magnet for 15 sec.
15. Collect the supernatant and transfer to a clean microtube. The supernatant contains the eluted RNA.

### Example 2: In Vitro Transcription (IVTS)

Plasmid DNA comprising a target gene insert conjugated to a T7 promoter was provided as template and the promoter insert sequence was amplified by PCR using a pair of primers with a biotinylated forward primer to provide a biotin moiety upstream of the T7 promoter sequence as shown in the first step of FIG. 1.

The biotinylated PCR product was immobilized on Dynabeads™ M-280 Streptavidin as follows:
1. Prepare the Immobilization mixtures described in the Table 5.
2. Transfer 100 μL (1 mg) of re-suspended Dynabeads™ M-280 Streptavidin to three 1.5 mL tubes.
3. Place on the magnet and remove the supernatant.
4. Wash once by re-suspending the beads in 100 μL of 1×StA Bind/Wash buffer. Place on the magnet and discard the supernatant.
5. Re-suspend the beads in 80 μL of the Immobilization Mix prepared in step 1.
6. Incubate for 15 min at RT on ThermoMixerC at 1000 rpm.
7. Place the tube on the magnet.
8. Wash the DNA-bead complex by re-suspending in 200 μL of 1×StA Bind/Wash buffer or Stringent Wash buffer, as described in Table 5.
9. Place the tube on the magnet and remove the supernatant.
10. Repeat steps 8-9 twice (total of three washes).
11. Wash the DNA-bead complex by re-suspending in 200 μL of 10 mM Tris HCl pH 7.5.
12. Place on the magnet and remove the supernatant.
13. Repeat steps 11-12 once (total of two washes).
14. Re-suspend the bead-DNA complex in 200 μL of 10 mM Tris HCl pH 7.5 and store at +4° C.

TABLE 5
Immobilisation mixtures

| Immobilisation | PCR product | H₂O | 2× StA Bind/Wash | Wash |
|---|---|---|---|---|
| # | (μL) | (μL) | buffer (μL) | Buffer |
| 1 | 10 | 30 | 40 | 1X StA B/W |
| 2 | 10 | 30 | 40 | Stringent |
| 3 | 5 | 35 | 40 | Stringent |

Solid phase in vitro transcription (IVTS) was performed as follows:
1. Place the bead-DNA complex on the magnet and discard the supernatant.
2. Wash the bead-DNA complex by re-suspending in 200 μL of 10 mM Tris HCl, pH 7.5.
3. Make the IVTS reaction mix: MEGAscript scaled up to 50 μL of reaction volume (Table 6).
4. Place the bead-DNA complex on the magnet and discard the supernatant.
5. Re-suspend the bead-DNA complex in 50 μL of MEGAscript reaction mix.
6. Incubate for 3 hours at 37° C. on the ThermomixerC at 1500 rpm.
7. Place the beads on the magnet and transfer the supernatant containing the in vitro transcript to a new, RNase-free tube. Heat the sample at 70° C. for 5 min and chill on ice.
8. Split each crude IVTS into two equal volumes (2×25 μL).
9. Wash the streptavidin-beads-DNA complex once with 200 μL of 10 mM Tris HCl pH 7.5.
10. Wash the beads-DNA complex, once with 200 μL of 1×StA Bind/Wash buffer.
11. Re-suspend in 200 μL of 1×StA Bind/Wash buffer. Store the beads at +4° C. for subsequent use.

TABLE 6
MEGAscript mix

| | Volume for | Volume for |
|---|---|---|
| Reagents | 1 rxn | 4.5 rxn |
| 10X T7 Reaction buffer* | 5 | 22.5 |
| ATP | 5 | 22.5 |
| CTP | 5 | 22.5 |
| GTP | 5 | 22.5 |
| UTP | 5 | 22.5 |
| Nuclease-free water | 20 | 90 |
| T7 Enzyme mix | 5 | 22.5 |

*10X T7 Reaction buffer: 0.4M Tris-HCl, pH 8.0 (+20° C.), 60 mM MgCl₂, 100 mM dithiothreitol, 20 mM spermidine

### Example 3: Determination of DNA and Protein Contamination

DNA template and protein contaminants were quantified by qPCR and fluorescence-based assay, respectively.

Contamination by DNA templates was quantified using qPCR, as follows:
1. Thaw all reagents, mix by vortexing and then centrifuge the tubes briefly. Keep on ice.
2. Prepare one PCR reaction mix before transferring it to the reaction plate.

| | | |
|---|---|---|
| 1. | 2X SYBR Green PCR Master Mix (2X) | 10 μL × n rxn = 10n μL |
| 2. | Forward primer (10 μm) | 1 μL × n rxn = n μL |
| 3. | Reverse primer (10 μm) | 1 μL × n rxn = n μL |
| 4. | H₂O | 8.8 μL × n rxn = 8.8n μL |

3. Transfer 19 μL of the PCR reaction mix to each well of the 96-well PCR plate.
4. Add 1 μL of the diluted mRNA or standard sample.
5. Centrifuge the plate, keep in dark.
6. Set-up the instrument protocol for qPCR according to Table 7.

TABLE 7
Thermal cycling conditions for qPCR

| | | | CYCLE (50 cycles) | | Dissociation | | | |
|---|---|---|---|---|---|---|---|---|
| Step | HOLD | HOLD | Denature | Anneal/Extend | (1 cycle) | | | |
| Time | 2 min | 10 min | 15 sec | 1 min | 15 sec | 1 min | 15 sec | 15 sec |
| Temp | 50° C. | 95° C. | 95° C. | 60° C. | 95° C. | 60° C. | 95° C. | 60° C. |

Protein contaminants were quantified in a fluorescence based assay using the Qubit Protein Assay kit, as follows:
1. Set up the required number of 0.5 mL tubes for standards and samples (3 standards and n samples).
2. Label the tube lids.
3. Prepare the working solution: dilute the Protein reagent 1:200 in Protein Buffer (in a plastic tube). Note: The final volume in each tube must be 200 μL (prepare (3+n)×200 μL).
4. Add 190 μL of working solution to each of the tubes used for standards.
5. Add 10 μL of each standard to the appropriate tube, mix by vortexing 2-3 sec. Be careful not to create bubbles.
6. Add 190 μL of working solution to individual assay tubes and add 10 μL of each sample (the final volume in each tube after adding sample is 200 μL).
7. Mix by vortexing 2-3 sec.
8. Allow all tubes to incubate at RT for 15 min.
9. Proceed to “Reading standards and samples” on the Qubit fluorometer.
   a. On the home screen, press Protein. The “Read standards” screen is displayed. Press Read standards to proceed.
   b. Insert the tube containing Standard #1 into the sample chamber, close the lid, the press Read standard. When the reading is complete (3 sec), remove Standard #1.
   c. Insert the tube containing Standard #2 into the sample chamber, close the lid, the press Read standard. When the reading is complete (3 sec), remove Standard #2.
   d. Insert the tube containing Standard #3 into the sample chamber, close the lid, the press Read standard. When the reading is complete (3 sec), remove Standard #3.
   e. Press Run samples.
   f. On the assay screen, select the sample volume and units:
      i. Press + or − buttons on the wheel to select sample volume added to the assay tube (10 μL)
      ii. From the dropdown menu, select the units for the output sample concentration
   g. Insert a sample tube into the sample chamber, close the lid, the press Read tube. When the reading is complete (3 seconds), remove the sample tube.
   h. Repeat step g until all samples have been read. Read each sample three times.

### Example 4: RNA Precipitation on Carboxylic Acid Beads

Several RNA extraction methods using polyethylene glycol (PEG) as a precipitating agent were developed (Lever M. A. et al., Frontiers in Microbiology, May 2015, Volume 6, Article 476). Here, the combination of PEG with a salt was tested. MyOne™ Carboxylic Acid beads were suspended in either 0.5 M NaCl, 5% PEG or 0.5 M NaCl, 10% PEG did not show any aggregation (FIGS. 4A and 4B, respectively).

Whereas a 5% v/v PEG-containing RNA binding buffer was not sufficient to precipitate the RNA (FIGS. 5A and B), the 10% v/v PEG-containing buffer enabled the expected 2.5-fold up-concentration (FIG. 5A) and the recovery of 94% of the starting material (FIG. 5B). Moreover, both A₂₆₀/A₂₃₀ and A₂₆₀/A₂₃₀ ratios were around 2 when the RNA was precipitated in the 10% v/v PEG-containing buffer (FIGS. 5C and D), indicating that 10% v/v PEG combined with a salt efficiently precipitated RNA without contaminant's carryover.

### Example 5: Preparation of a Large Batch of RNA

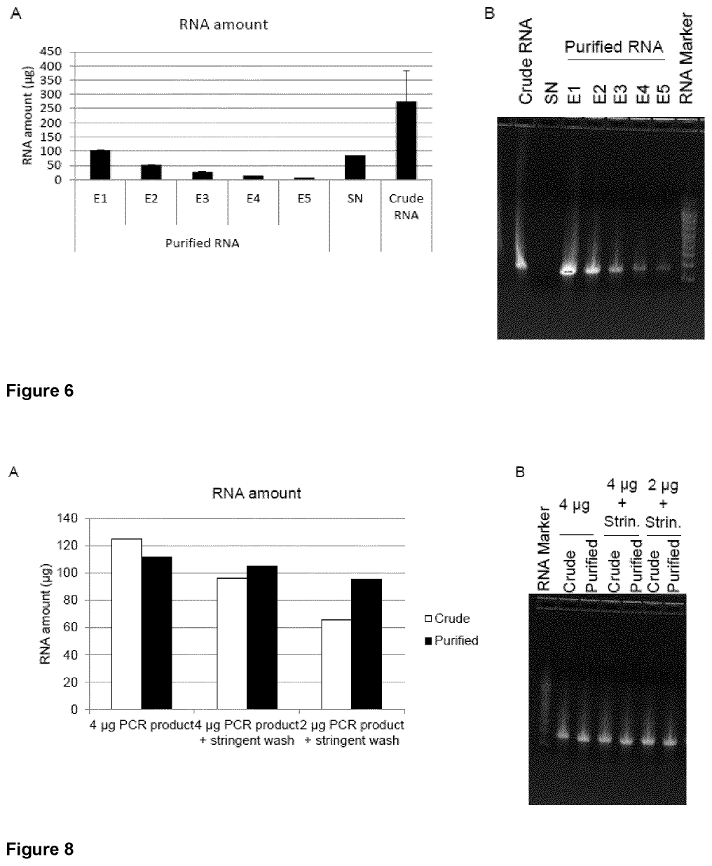

A large batch of IVTS RNA was prepared according to the protocol described in Example 2. The batch was then split into two equal fractions: one fraction was kept as is and represented the crude IVTS RNA fraction, the other fraction was further purified on Dynabeads™ Oligo(dT)₂₅ and represented the purified IVTS RNA fraction. Five different elution fractions (E1, E2, E3, E4 and E5) were collected after purification on Oligo(dT)₂₅-coupled beads and the RNA content was measured. The total of the RNA content of the five eluates was close to the RNA amount of the crude IVTS RNA, indicating that only a small fraction of RNA was lost during the purification (FIG. 6A). Moreover, the integrity of the crude and purified RNA was satisfactory according to the migration pattern obtained in a denaturing agarose gel (FIG. 6B).

### Example 6: RNA Recovery and Up-Concentration

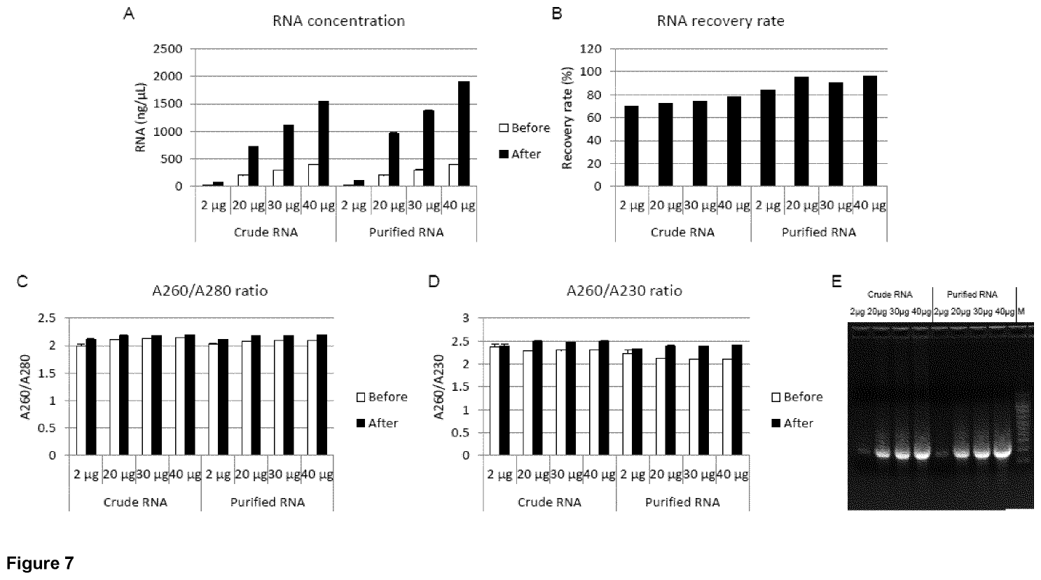

Varying amounts of the crude or purified IVTS RNA samples were precipitated on 800 μg of Dynabeads MyOne Carboxylic acid beads, in accordance with the RNA protocol of Example 1. Both RNA types were efficiently up-concentrated regardless the amount of starting material (FIG. 7A). The recovery rates were around 74% for the crude IVTS RNAs and around 94% for the purified IVTS RNAs, suggesting that some contaminants present in the crude samples hindered the precipitation on the beads (FIG. 7B). Alternatively, the RNA detected in the SN fraction may represent shorter poly(A)-free RNA molecules. Both A₂₆₀/A₂₃₀ and A₂₆₀/A₂₃₀ ratios were around 2 in all samples, showing a satisfactory purity level of the RNA (FIGS. 7C and D). Moreover, the up-concentrated RNAs were evaluated by electrophoresis and showed no degradation (FIG. 7E).

### Example 7: Calibration of the Amount of Biotinylated DNA Template

In an attempt to decrease the DNA contamination in the IVTS RNA sample, the amount of biotinylated PCR product used for immobilization on Dynabeads™ M-280™ Streptavidin beads was calibrated. Either 4 or 2 μg of PCR product was used for immobilization and the washing step included or did not include a nonionic detergent (stringent wash). The amount of RNA in the crude and purified samples was 10-15% lower when beads were washed in the presence of a detergent than with the usual wash buffer (FIG. 8A). The beads bound to 2 μg of DNA led to the generation of 50% less crude RNA and 15% less purified RNA than the beads bound to 4 μg of DNA (FIG. 8A). These results indicate that the stringent wash had a negligible effect on the efficiency of the in vitro transcription, whereas the use of twice less DNA for IVTS template generation decreases the RNA yield. Moreover, the integrity of the crude and purified RNA samples was satisfactory according to the migration pattern in a denaturing agarose gel (FIG. 8B).

### Example 8: RNA Precipitation on Carboxylic Acid Beads

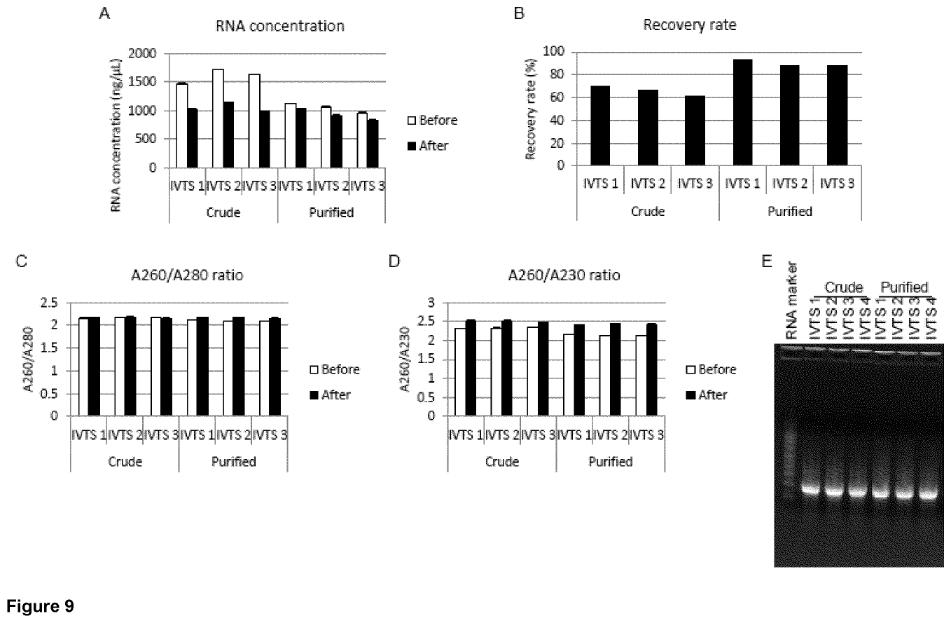

Those different crude and purified IVTS RNA samples were then used for RNA precipitation on Dynabeads™ MyOne™ Carboxylic Acids beads, in accordance with the RNA protocol of Example 1. Around a third of the crude IVTS RNA samples were lost after precipitation, whereas the precipitation of the purified IVTS RNA samples was almost total (FIG. 9A). Likewise, the recovery rates were around 66% for the crude samples and around 90% for the purified ones (FIG. 9B). This result is consistent with the results illustrated in FIG. 7B. Both A₂₆₀/A₂₃₀ and A₂₆₀/A₂₃₀ ratios were around 2 in all samples, showing a satisfactory purity level of the RNA (FIGS. 9C and D). Moreover, the eluted RNAs after precipitation were evaluated by electrophoresis and showed no degradation (FIG. 9E).

### Example 9: DNA Contamination

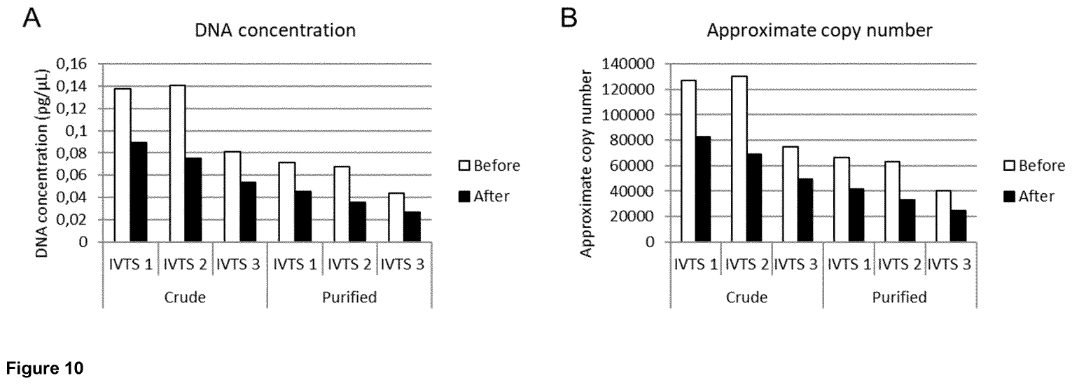

Next, the presence of DNA template in the IVTS RNA samples was measured by qPCR, in accordance with the qPCR assay of Example 3. The amplification curves and the melting curves (not shown) indicated that the amplification efficiency was similar for all samples and that the PCR reaction was specific, respectively. Based on the standard curve (not shown), the DNA concentration was calculated. The DNA concentration ranged from 0.02 to 0.14 μg/μL depending on the condition (FIG. 10A). The DNA contamination was approximately twice lower in the purified IVTS RNA samples than in the crude ones (FIG. 10A). Interestingly, the DNA contamination was noticeably reduced in the IVTS samples after precipitation, compared to the results for contamination before (FIG. 10B), indicating that the TEG and NaCl conditions provide preferential precipitation of RNA.

### Example 10: Protein Contamination

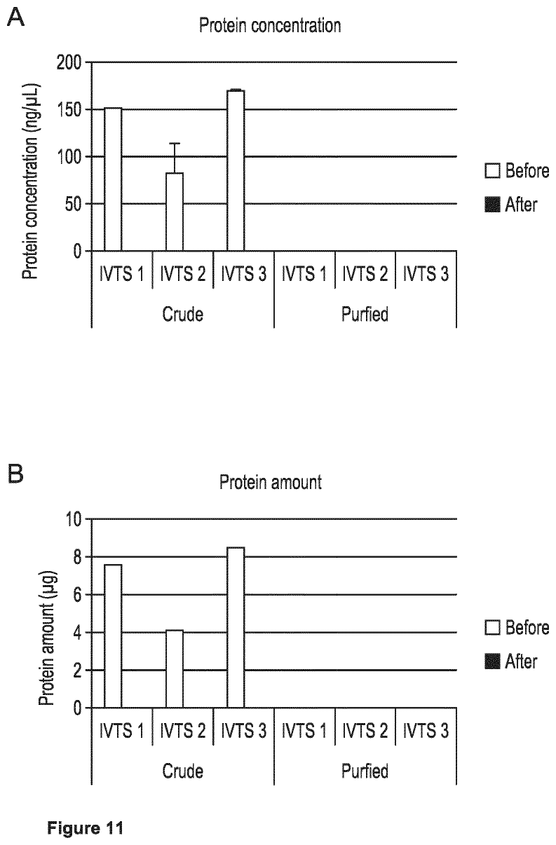

The presence of protein contamination was assessed in the crude and purified IVTS RNA samples, in accordance with the fluorescence-based assay of Example 3. The non-diluted IVTS samples were subjected to a fluorescence-based assay on the Qubit 3 instrument. Only the crude IVTS RNA samples before precipitation contained between 100 and 150 ng/μL of protein (FIG. 11A), which corresponded to 4 to 8 μg (FIG. 11B). All purified IVTS RNA samples, as well as the crude IVTS samples after precipitation, had no detectable protein content (FIGS. 11A and B), indicating that the RNA precipitation workflow did not carryover protein contaminants.

### Example 11: Effect of Order of Reagent Addition and Drying of Beads

To test whether the order of reagent addition and drying of the beads with ethanol affects handling and yield of mRNA, during generic capture clean-up of in vitro transcribed mRNA the experimental set up shown in Table 8 was investigated. A crude sample of in vitro transcription mix diluted to 3 μg/μL mRNA was used in this example.

TABLE 8
Experimental setup
Preparation of 200 mL (1.5X) RNA binding buffer:

| Final conc | | | Mixing of stock solutions for 100 mL: | | | |
|---|---|---|---|---|---|---|
| TEG | NaCl | Tris-HCl | 99% TEG | 5 M NaCl | 1M Tris pH 8 | H₂O |
| (% v/v) | (5 M) | pH8 | | | | Nuclease- |
| | | | | | | Free |
| 60% | 1.5 M | 37.5 mM | 121.2 mL | 60 mL | 7.5 mL | 11.3 mL |

Preparation of 200 mL washing buffer.

| Final conc | Mixing of stock solutions for 200 mL: | | | | | |
|---|---|---|---|---|---|---|
| EtOH (% v/v) | 99.8% EtOH | H₂O | | | | |
| | | Nuclease- | | | | |
| | | Free | | | | |
| 70% | 140 mL | 60 mL | | | | |
| Samples | 1 | 2 | 3 | 4 | 5 | 6 |
| Bead | MyOne | MyOne | MyOne | MyOne | MyOne | MyOne |
| content | COOH | COOH | COOH | COOH | COOH | COOH |
| Parallels | 1A 1B | 2A 2B | 3A 3B | 4A 4B | 5A 5B | 6A 6B |
| Drying | No dry | 2.5 min | 5 min | 10 min | Full dry | 10 min |
| procedure | | | | | (37° C.) | |
| RBB add to | Beads | Beads | Beads | Beads | Beads | Beads + |
| (adding order) | | | | | | RNA |

| | |
|---|---|
| Beads (μg) | 300 |
| mRNA (μg) | 300 |

| | |
|---|---|
| Calculated volumes: | |
| μL Beads, 10 mg/mL | 30 |
| μL mRNA | 100 |
| μL RBB (1.5X) | 200 |
| Total reaction volume | 300 |

A. Generic Capture onto Dynabeads™ MyOne™ Carboxylic Acid Beads:
1. Thoroughly mix the beads, vortex for 10 seconds and place on a roller for 20 min at RT.
2. Pipette the given volume of bead suspension into clean tubes according to Table 8.
3. Place the tubes on the magnet for 30 seconds and discard the supernatant.
4. Add 200 μL 1.5×RBB according to Table 8 and pulse vortex.
5. Add the given volume RNA and nuclease-free water according to Table 8.
6. For sample 6, reverse the order of step 4 and 5.
7. Mix by inverting the tubes 3-4 times and incubate for 10 min at RT on thermo mixer 1000 rpm RT.
8. Quickly spin down and place the tube on the magnet for 30 seconds and discard the supernatant.
9. Add 500 μL of WB. Resuspend the beads by vortexing.
10. Place the tube on the magnet for 30 seconds and discard the supernatant.
11. Repeat steps 8-9 once (total of two washes). Make sure that there are no droplets left on the wall or lid of the tube (spin down or by pipette).
12. Let the beads dry at RT for 10 min on the magnet (except for full dry, 37° C.) according to Table 8.
13. Remove the magnet and add 100 μL of EB. Re-suspend the beads by vortexing for 20 seconds and incubate at 65° C. for 5 min on Thermomixer 1000 rpm.
14. Vortex the beads for 10 seconds and place the tubes on the magnet for 30 seconds.
15. Collect the supernatant and transfer to clean tubes.

B. Analysis:
1. Measure RNA concentrations on the Qubit (see below)
2. Run RNA stock solution and samples on a gel:
3. Take 2 μL of the RNA stock solution and samples and add 8 μL dH2O and 10 μL 2× Gel loading buffer II.
4. Take 10 μL of RNA Millennium ladder and add 10 μL of 2× Gel loading buffer II.
5. Heat all samples at 70° C. for 10 min and chill on ice.
6. Load 20 μL of the RNA samples and ladder: include crude, to see the purified difference

B1. RNA—Qubit Mix RNA BR

Dilute stock solution and purified IVTS RNA samples 1:100 in NanoPure water: 10 μL+990 μL NanoPure water.
a) 2 replicates of 6 samples+2 standards=12 qubit samples total (make for 13 samples for extra volume)
b) Prepare working solution: (calculate 200 μL per sample=final volume: 2600 μL)
c) Dilute Qubit reagent 1:200 (For 2.6 mL: 13 μL Qubit RNA BR reagent+2587 μL Qubit RNA BR buffer)
d) Add 190 μL Qubit working solution to each of the standard tubes and sample tubes
e) Add 10 μL of each standard and 100× diluted sample
f) Mix by vortexing and spin down
g) Incubate at RT for 3 min
h) Read

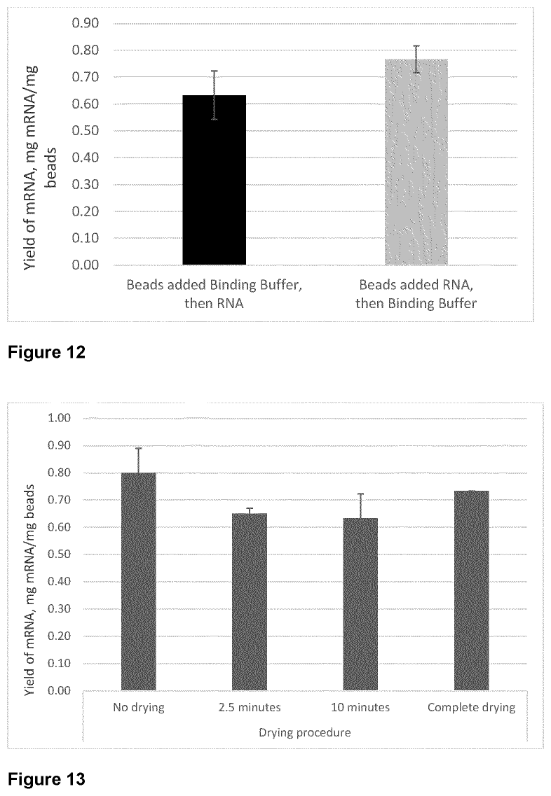

As shown in FIG. 12, mixing of beads and RNA before addition of the RNA Binding Buffer reduced bead aggregation, allowed for easier dispersion by resuspension and increased yield of RNA. In addition, ethanol drying of the beads was not needed for efficient mRNA elution. The sample with no drying gave equal or higher yield of mRNA as compared to the completely dried sample (FIG. 13).

The reagents employed in the examples are commercially available or can be prepared using commercially available instrumentation, methods, or reagents known in the art. All references cited herein are incorporated by reference in their entireties. The foregoing examples illustrate various aspects of the invention and practice of the methods of the invention. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the appended claims.

### Example 12: Comparison of mRNA Capture Using Different Carboxylic Acid Activated Beads

Different beads functionalized with carboxylic acid (as listed in Table 9) were used to purify in vitro transcribed mRNA.

TABLE 9
carboxylic acid-activated beads

| Bead | Size | | Used amounts (μg) |
|---|---|---|---|
| Dynabeads ™ MyOne ™ Carboxylic Acid | 1 | μm | 100, 200, 300 |
| CA-1 | 1 | μm | 100, 200, 300 |
| CA-2 | 1 | μm | 100, 200, 300 |
| Dynabeads ™ M-270 Carboxylic Acid | 2.8 | μm | 100, 200, 300 |
| CA-3 | 5 | μm | 200, 300, 400, |
| | | | 500, 600 |

In vitro transcribed mRNA produced as described in Example 2 was diluted to 3 μg/μL and 100 μL of this mRNA sample was added to different amounts of beads as indicated in Table 9. The bead/RNA suspension was then mixed with 200 μL RNA binding buffer following the protocol as described in Example 11. Results were analyzed by Qubit, as described in Example 11.

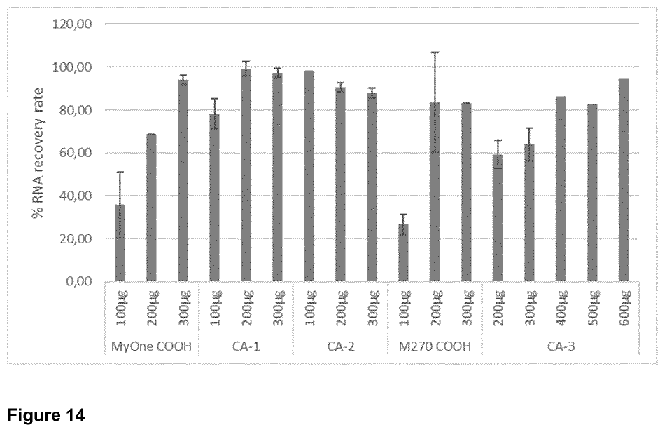

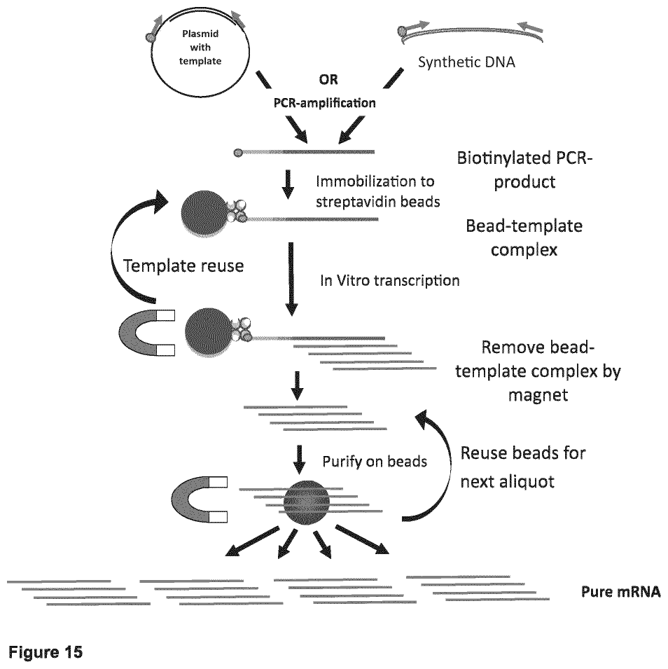

FIG. 14 shows the normalized results from the experiments summarized in Table 10 below (300 μL binding volume, 300 μg RNA).

TABLE 10
Experimental setup

| Experiment No. | Beads | Bead input (μg) |
|---|---|---|
| 1 | Dynabeads ™ MyOne ™ Carboxylic Acid, CA-1, CA- | 200 |
| | 2, Dynabeads ™ M-270 Carboxylic Acid, CA-3 | |
| 2 | Dynabeads ™ MyOne ™ Carboxylic Acid, CA-1, CA- | 300 |
| | 2, Dynabeads ™ M-270 Carboxylic Acid, CA-3 | |
| 3 | Dynabeads ™ MyOne ™ Carboxylic Acid, CA-1, CA- | 100 |
| | 2, Dynabeads ™ M-270 Carboxylic Acid | |
| 4 | CA-3 | 400, 500, 600 |

The data shown in FIG. 14 illustrates that all tested beads capture the RNA with more than 80% recovery, when sufficient bead surface area is provided. For example, for the largest diameter beads (the CA-3 beads), a greater mass of beads was used to obtain a surface area similar to the surface area provided with lower masses of the smaller beads.

### Example 13: Solid Phase In Vitro Transcription and Cleanup Protocol

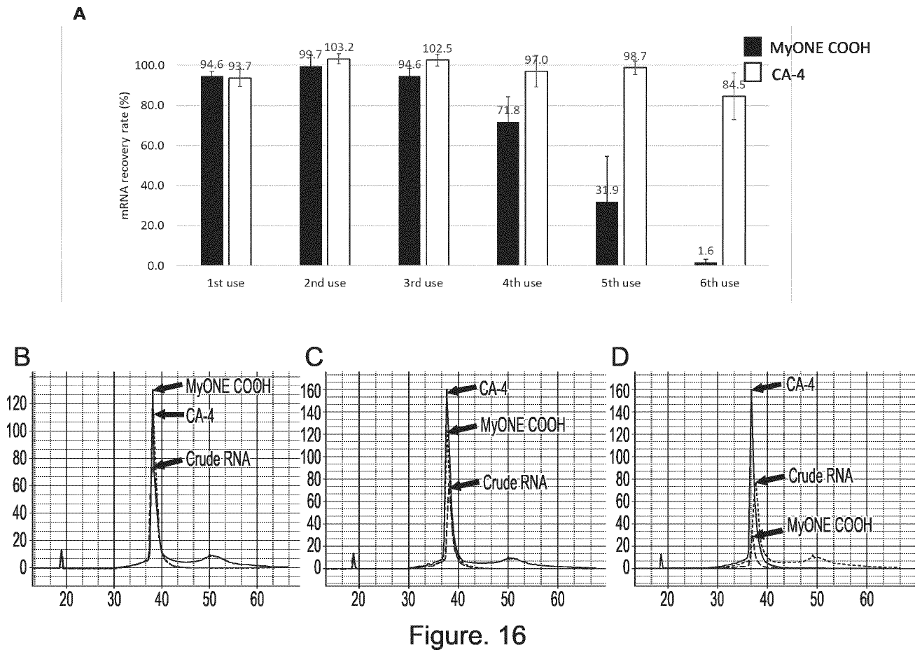

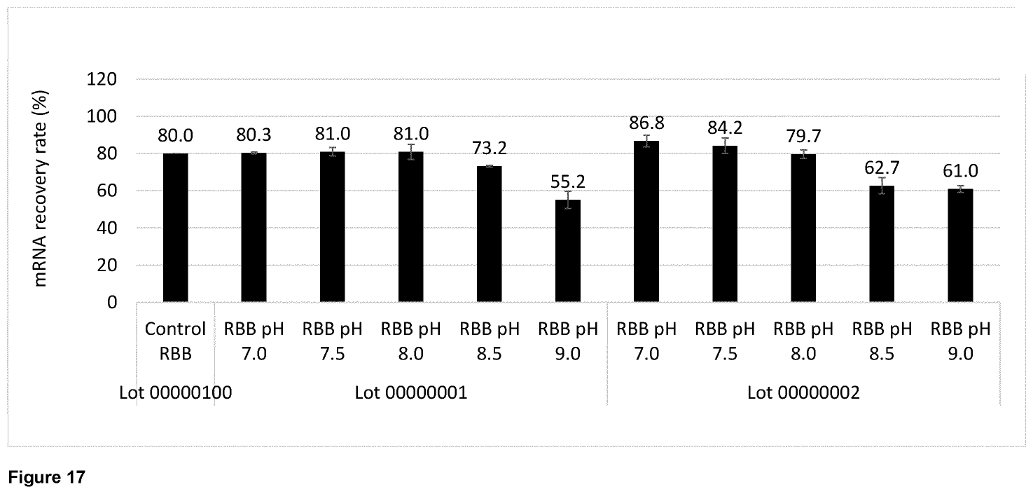

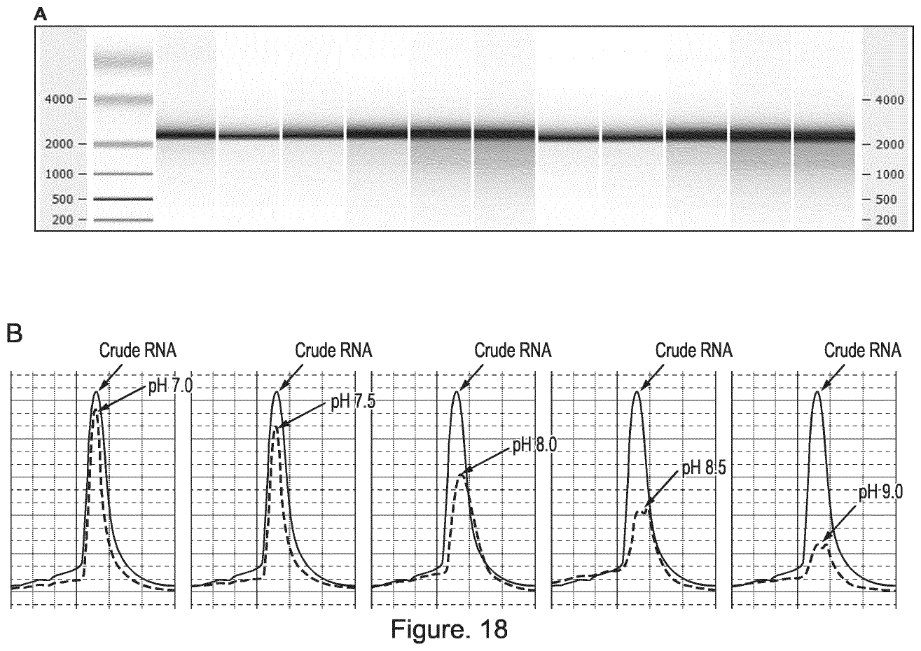

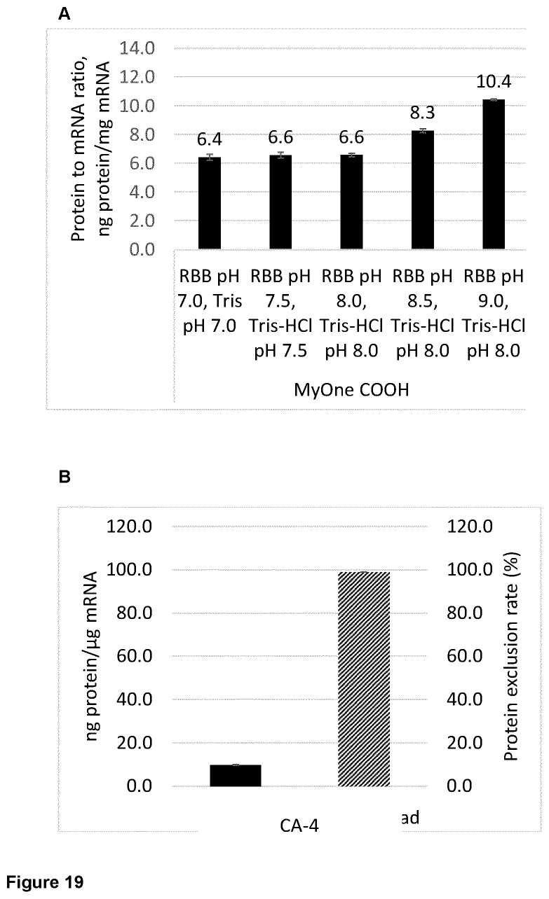

This protocol was used to generate the data illustrated in FIGS. 16 to 19. As a starting point, use 1 mg Dynabeads™ Streptavidin beads+1 μg biotinylated DNA (PCR product).

### Materials and Methods

Dynabeads™ Streptavidin Beads
1×Streptavidin BW-buffer (5 mM Tris-HCl (pH 7.5) 0.5 mM EDTA 1 M NaCl)
Nuclease free water
10 mM Tris pH 7-8
TE pH 7-8
Vortexer
Roller
Thermomixer

### Immobilization of Biotinylated PCR-Product (Template) to Dynabeads™ Streptavidin

Prepare a biotinylated PCR product containing the T7-promoter upstream of the UTR and ORF, optionally with a defined polyA-tail in the end. The forward primer must be biotinylated and have a distance to the T7 promoter of at least 50-100 bp. Dilute the PCR product to 20 ng/μL in 1×Streptavidin BW-buffer.

### Immobilization of PCR-Product to Streptavidin Beads (1 mg Dynabeads™ Streptavidin)

An overview of the process in set out in Table 11.

TABLE 11
Overview:

| | Biotinylated | | |
|---|---|---|---|
| | PCR product | | |
| Dynabeads ™ | 1 μg (in 1× StA | Final NA | Final bead |
| Streptavidin beads | BW-buffer) | amount/bead | concentration |
| 100 μl = 1 mg | 50 μl of 20 ng/ | 1 μg/mg | 10 mg/ml |
| | μl = 1 μg | | |

Preparation of Streptavidin Beads:
1. Resuspend the Dynabeads™ Streptavidin completely by vortexing
2. Place the Dynabeads™ Streptavidin on a roller for at least 20 min
3. Transfer 100 μL (=1 mg) resuspended Dynabeads™ Streptavidin to an RNase free tube
4. Place on magnet for 1 minute and remove supernatant
5. Wash beads once in 100 μL 1×StA BW buffer, by resuspending using a pipette or brief vortexing
6. Place on magnet for 1 minute
7. Remove supernatant
8. Resuspend beads in 50 μL 1×StA BW buffer

Immobilization:
9. Add 50 μL (1 μg diluted in 1×StA BW buffer) biotinylated PCR-product to the washed and resuspended Dynabeads™ Streptavidin
10. Incubate for 30 min at Room Temp (RT) on Thermomixer 1500 RPM
11. Place on magnet for 1 minute and remove supernatant (*optional: quantify how much unbound remains in this supernatant) *Optional—from step 11: Measure remaining DNA template in supernatant after immobilization by 1×dsDNA HS assay kit.
12. Wash DNA-bead complex by resuspending in 100 μL TE-buffer pH8, using a pipette or brief vortexing
13. Place on magnet for 1 minute and discard supernatant
14. Repeat wash three times, a total of 4 washes. For last wash step, do not discard supernatant.

Solid-Phase In Vitro Transcription of Immobilized Template

Materials and methods:
Nuclease free water
MEGAscript kit, (Thermo Fisher Scientific, cat #AMB1334).
10 mM Tris HCl pH8
1×StA BW-buffer (5 mM Tris-HCl (pH 7.5) 0.5 mM EDTA 1 M NaCl)
Thermomixer

As template: 1 mg Streptavidin beads with 1 μg biotinylated DNA template immobilized (from step 18 above).

Make the IVTS-Reaction mix: MEGAscript scaled up to 100 μL reaction is shown in Table 12 (Note all reagents should be kept on ice while using the kit)

TABLE 12
Scaling up transcription

| | Kit manual: | Scale up (**): 5× for |
|---|---|---|
| | 1× for 20 μl reaction | 100 μL rxn volume |
| Reagents | volume (μL)/rxn | (μL)/rxn |
| Nuclease free | 8 μl (*) | 40 μL |
| dH₂O, from kit | | |
| ATP | 2 | 10 |
| CTP | 2 | 10 |
| GTP | 2 | 10 |
| UTP | 2 | 10 |
| 10× rxn buffer | 2 | 10 |
| Enzyme mix | 2 | 10 |
| Template = T | Bead pellet (*) | Bead pellet |
| Total volume | 20 | 100 |

(*) Template on beads → neglectable volume of liquid around bead pellet does not need to be subtracted
(**) The reaction is directly scalable, by increasing all components linearly.

The steps are as follows:
1. Place DNA-bead complex from step 14 above, on magnet and discard supernatant
2. Wash DNA-bead complex by resuspending in 200 μL 10 mM TrisHCl, pH8
3. Place DNA-bead complex on magnet, discard supernatant
4. Resuspend DNA-bead complex in 100 μL MEGAscript reaction mix
5. Incubate for 1-3 hours (100 μL) at 37° C., Thermomixer at 1500 RPM.
6. Place beads on magnet, and transfer the supernatant containing the in vitro transcript to a new, RNase free tube. Place on ice prior to analysis or freeze at −70° C.

Measure mRNA concentration, using a Qubit RNA Assay kit.

A 100 μL, 2 hours reaction typically yields 4 μg/μL mRNA, giving up to 400 μg per reaction.

The bead-template complex may be reused up to 6 times in successive in vitro transcription reactions, simply by transferring the supernatant to a new tube and adding new MEGAscript mix, or store at 4° C., for later use.

Storage and Reuse of Reuse of the Bead-DNA Complex from the Reactions:
a. Wash the DNA-bead complex once with 200 μL 10 mM Tris HCl pH8
b. Wash the DNA-bead complex, once with 200 μL 1×StA BW buffer.
c. Resuspend in 200 μL 1×StA BW buffer. Store at +4° C. for re-use later in vitro transcription.
   mRNA Cleanup by Generic Capture onto Dynabeads MyOne™ Carboxylic Acid (COOH) Beads
1. Thoroughly mix Dynabeads MyOne™ COOH, vortex for 10 seconds and place on a roller for 20 min at RT.
2. Pipette 30 μL=300 μg (concentration: 10 mg/mL) bead suspension into clean nuclease free tubes.
3. Place the tubes on the magnet until the solution looks clear (approximately 30-60 seconds) and discard the supernatant.
4. Wash once in 100 μL nuclease free water and place on magnet, discard supernatant.
5. Add 100 μL mRNA solution containing up to 150 μg RNA and mix until beads are well resuspended.
6. Add 200 μL 1.5×RBB (RNA Binding Buffer), and mix by pipetting, until homogenous suspension
7. Incubate for 10 minutes on a Thermomixer 1000 rpm room temp.
8. Place the tube on the magnet until the solution looks clear (approximately 2-3 minutes, since the solution is quite viscous) and discard the supernatant.
9. Add 500 μL of WB (70% EtOH in nuclease free water). Resuspend the beads by vortexing.
10. Place the tube on the magnet until the solution looks clear (approximately 30-60 seconds) and discard the supernatant.
11. Repeat 9-10 twice, a total of 3 washes. Be sure to remove all residual WB.
12. Let the beads dry at RT for 10 min while on the magnet.
13. Remove the magnet and add 100 μL of Elution Buffer. (*)TE buffer pH7-8, 10 mM TrisHCl pH7-8 or nuclease free water. Re-suspend the beads by pipetting and incubate at 65° C. for 5 min on Thermomixer 1000 rpm. After incubation, short spin of tube to collect liquid from lid is optional.
14. Place the tube on the magnet until the solution looks clear (approximately 30-60 seconds).
15. Collect the supernatant and transfer to clean nuclease free tubes and place on ice or freeze.

### Example 14: Large Scale mRNA Production and Purification and Reuse of Beads

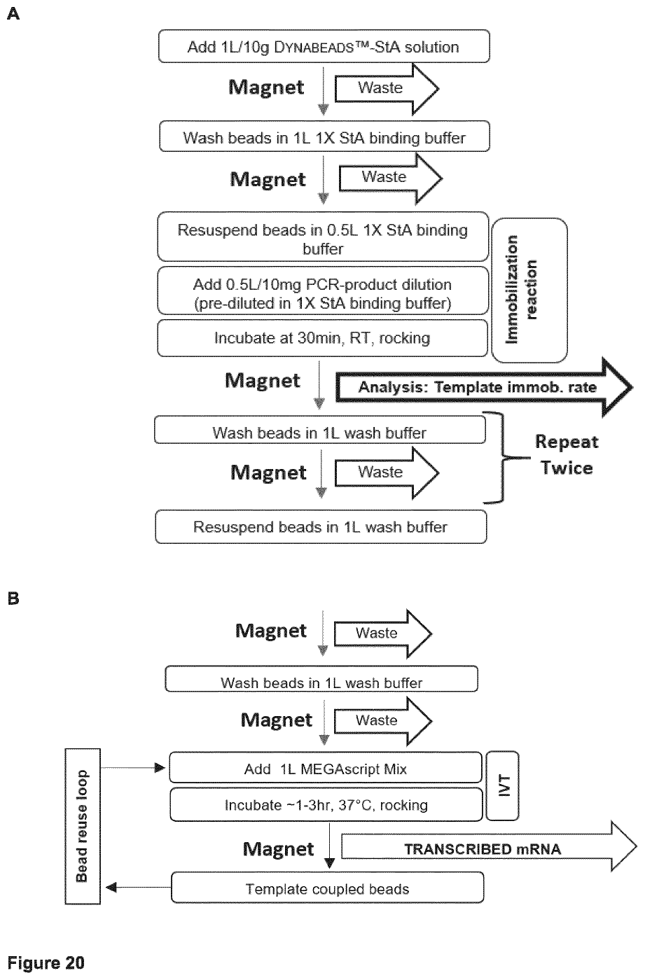

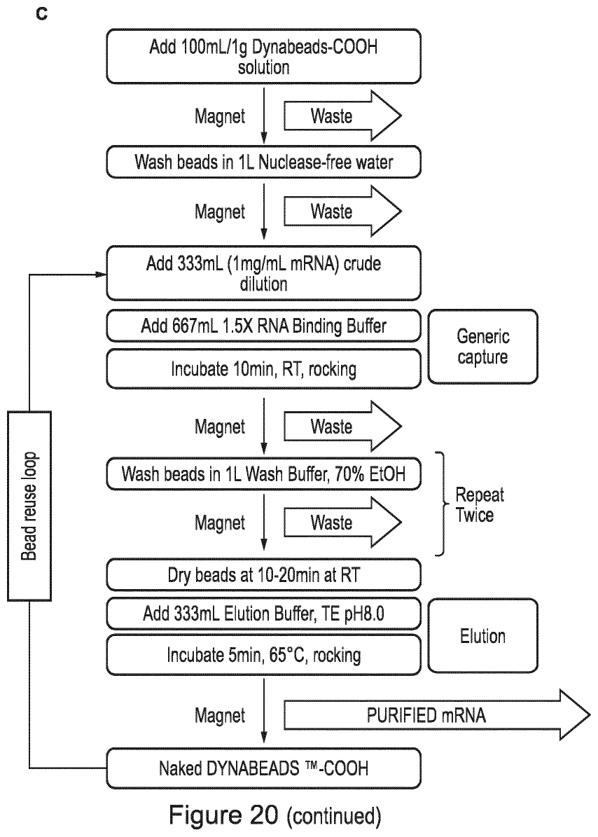

For the initial set up, the buffers and reagents were loaded in a glass bioreactor. Alternatively, bags may be used. Dynabeads™ MyOne™ Streptavidin C₁ were thoroughly resuspended prior to the transfer to the reactor. The beads were vortexed until they were properly resuspended and placed on a roller for 20 minutes. Also, 10 mg of biotinylated template DNA/PCR-product were diluted in 1×Streptavidin Binding and Washing Buffer (5 mM Tris-HCl (pH 7.5) 0.5 mM EDTA, 1 M NaCl) up to a total volume of 0.5 liters (L). Outlines of the workflows described in this example are shown in FIG. 20 A-C.

Part 1: Template Immobilization on Dynabeads™ MyOne™ Streptavidin C1 (Thermo Fisher Scientific, Cat. No. 65002), Beads

10 g of Dynabeads™ MyOne™ C1 Streptavidin beads (1 L, 10 mg/mL) were transferred to a reactor. A magnet was applied until the supernatant was completely translucent and the supernatant was removed and transferred to waste. After a wash step applying a magnet the magnet was removed and 1 L of 1× Streptavidin Binding Buffer was transferred to the reactor and mixed for 1 minute with gentle rocking. A small volume of air was added to the bioreactor to aid with the mixing. A magnet was then applied until the supernatant was completely translucent and the supernatant was transferred to waste. The magnet was then removed and another 0.5 L of 1× Streptavidin Binding Buffer was transferred to the reactor and mixed for 1 minute with gentle rocking. Again, a small volume of air was added to aid with the mixing. 0.5 L of 10 mg biotinylated template DNA dilution (pre-diluted in 1× Streptavidin Binding Buffer) was then added to the reactor and incubated for 30 minutes at room temperature with thorough rocking. A magnet was then applied until the supernatant was completely translucent and the supernatant was collected and stored at 4° C. The supernatant can be used to quantify the degree of biotinylated template DNA immobilized on the Streptavidin beads. After a washing step, the magnet was removed and 1 L of 1× Streptavidin Binding and Washing Buffer was transferred to the reactor and mixed for 1 minute with gentle rocking. Again, a small volume of air was added to the reactor to aid with the mixing. Then a magnet was applied until the supernatant was completely translucent and the supernatant was removed and transferred to waste. The washing step may be repeated several times (e.g. a total of 4 washes). The magnet was then removed and the bead-template complex resuspended in 1 L of Washing Buffer and a small volume of air was added to aid with the mixing. The workflow of part 1 is illustrated in FIG. 20A.

Part 2: Solid Phase In Vitro Transcription

The MEGAscript MIX was prepared according to manufacturer instructions and kept on ice prior to use. MEGAscript™ kit reagents were scaled up to using the ratios of components set out in Part 1. A magnet was applied until the supernatant was completely translucent and the supernatant was then removed and transferred to waste. To wash the beads, the magnet was removed and 1 L of Wash Buffer was transferred to the reactor and mixed for 1 minute with gentle rocking. Again, a small volume of air was added to aid with the mixing. A magnet was then applied until the supernatant was completely translucent and the supernatant was transferred to the waste. The magnet was then removed and 1 L of MEGAscript™ reaction mix was added to the reactor and incubated for 2 hours at 37° C. temperature with thorough rocking. Again, a small volume of air was added to aid with the mixing until the target concentration of transcribed mRNA was attained. A magnet was then applied until the supernatant was completely translucent and the transcribed mRNA was collected. As illustrated in FIG. 20 B, the template-coupled beads can be reused multiple times. For example, the MEGAscript™ reaction mix may be added to the reactor comprising the template-coupled beads 5 additional times in subsequent rounds of mRNA production.

Part 3: Generic Capture, mRNA Purification with Dynabeads™ MyOne™ Carboxylic Acid Beads

COOH beads (e.g. Dynabeads™ MyOne™ Carboxylic Acid beads (Thermo Fisher Scientific, cat. no. 65012) were thoroughly resuspended prior to use and placed on a roller for 20 minutes. 333 mg of IVT crude mRNA was then diluted in TE buffer of 10 mM Tris Buffer up to a total volume of 333 mL. 1 g Dynabeads™ MyOne™ Carboxylic Acid beads (100 mL, 10 mg/mL) were transferred to a reactor (reaction bags may also be used) and a magnet was applied until the supernatant was completely translucent and the supernatant was transferred to waste. After removal of the magnet, the beads were washed with 1 L of Nuclease-free water and allowed to mix for 1 minute with gentle rocking. A small volume of air was added to aid with the mixing. Again, a magnet was applied until the supernatant was completely translucent and the supernatant transferred to the waste. Upon removal of the magnet, 333 mL comprising 333 mg IVT crude mRNA mix (1 mg mRNA/mL dilution) was added to the reactor and mixed for 1 minute with thorough rocking and adding a small volume of air to aid with the mixing. 667 mL of 1.5×RNA Binding Buffer was then added to the reactor and incubated for 10 minutes at room temperature with thorough rocking and adding a small volume of air support the mixing. After applying a magnet the completely translucent supernatant was removed and transferred to waste. The magnet was then removed and 1 L of Wash Buffer was added to the reactor and allowed to mix for 1 minute with gentle rocking and again adding a small volume of air to support the mixing. A magnet was applied, and the completely translucent supernatant transferred to waste. The washing procedure was repeated two more times (i.e. a total of 3 washes). Beads were then dried at room temperature until completely dry. Drying may typically take about 10 to 20 minutes and may be achieved, for example, by using pump filtered air through the reactor. 333 mL of Elution Buffer was then added to the reactor and incubated for 5 minutes at 65° C. room temperature with thorough rocking and adding a small volume of air to support the mixing. Finally, a magnet was applied until the supernatant was completely translucent and the supernatant comprising the purified mRNA was then transferred to a collection compartment. As illustrated in FIG. 20C, the COOH beads may be reused several times in subsequent rounds to capture and purify transcribed mRNA.

### Example 15: Repeated Capture of mRNA with Carboxylic Acid Functionalized Beads

CA-4 beads with higher iron content and magnetic saturation were manufactured using methods as described in Examples 5 and 6 disclosed in U.S. Pat. No. 6,986,913 (the content of which is incorporated herein by reference in its entirety), with adaptation to incorporate additional iron oxide in the beads. 300 μg of Dynabeads™ MyOne™ Carboxylic Acid (MyOne™ COOH) and CA-4 beads each were then used and reused in six consecutive rounds of mRNA purification. 150 μg in vitro transcribed mRNA was added to the purification reaction in each round up to a binding volume of 300 μL, following the generic capture protocol disclosed in Example 13. As shown in FIG. 16A, mRNA yield dropped after 4 times reuse when MyOne™ COOH beads (black bars) were used, whereas mRNA yields were maintained even after 6 rounds of reuse when CA-4 beads with higher iron content were used (white bars). Likewise, as indicated by the RNA integrity data shown in FIG. 16B, the CA-4 beads maintained high performance through 5 rounds of mRNA purification, whereas MyOne™ COOH performance started to decline after the 3rd use.

The higher magnetic susceptibility of CA-4 beads as compared to MyOne™ COOH beads may be determined in accordance with a suitable assay, for example by using the measurement of saturation mass magnetization assay as described hereinabove under the heading “Assays”. Alternatively, such measurements may be performed in accordance with the method of Example 16.

### Example 16: Measurement of Magnetic Saturation of Magnetic Beads

Powder samples of magnetic particles were loaded onto a custom molded PVP powder holders that were then immobilized on a brass sample holder. The samples were first centered using 100 Oe magnetic field and then the measurements were run using a vibrating sample magnetometer working at 40 Hz vibrating frequency. The magnetization as function of applied magnetic field were carried out between the magnetic field values of +11000 Oe to −11000 Oe and back. A continuous mod was used for magnetization measurement with a magnetic field ramp rate of 10 Oe/sec. All measurements were carried out at room temperature. Raw data were then converted to SI-units in emu/g. The calculations were done using the following equations:

Magnetization=(moment (emu))/(weight (g))

H(A/m)=H(Oe)*1000/4π

1. A method of separating RNA from a sample, the method comprising:
a) providing
i) a sample comprising RNA;
ii) a binding solution comprising an oligoethylene glycol and a salt, the oligoethylene glycol comprising from about 2 to about 70 ethylene glycol units in linear arrangement; and
iii) a solid support having a hydrophilic surface;
b) contacting the sample with the binding solution and solid support, under conditions that allow binding of the RNA in the sample to the surface of the solid support, thereby providing a solid support with bound RNA in contact with residual solution; and
c) separating the solid support with bound RNA from the residual solution;
wherein, during the binding of the RNA in the sample to the surface of the solid support, the oligoethylene glycol is present at a concentration of at least about 35% v/v and the salt is present at a concentration of between about 1 M and about 2 M.

2. The method of claim 1, wherein during the binding, the oligoethylene glycol is present in a concentration of between about 35% v/v and about 50% v/v.

3. The method of claim 1 or claim 2, wherein the oligoethylene glycol comprises about 2 to about 20 ethylene glycol units in linear arrangement;
optionally wherein the oligoethylene glycol is or comprises triethylene glycol, tetraethylene glycol, pentaethylene glycol, hexaethylene glycol, or heptaethylene glycol.

4. The method of any preceding claim, wherein the oligoethylene glycol is or comprises tetraethylene glycol.

5. The method of any preceding claim, wherein the salt is selected from or comprises an alkali metal halide, or an alkaline earth metal halide;
optionally wherein the salt is selected from or comprises sodium chloride, potassium chloride, lithium chloride, magnesium chloride, calcium chloride and barium chloride.

6. The method of any preceding claim, wherein the salt is or comprises sodium chloride.

7. The method of any preceding claim, wherein the binding solution comprises buffer providing a pH of from about 6 to about 9;
optionally wherein the buffer provides a pH of about 7 to about 8.

8. The method of claim 7, wherein the buffer comprises or is selected from Tris, Tris/EDTA, PBS, citrate, 2-(N-morpholino)ethanesulfonic acid (MES), or water;
optionally wherein the buffer comprises or is selected from Tris, Tris/EDTA, PBS, or water.

9. The method of claim 7 or claim 8, wherein the buffer comprises Tris, optionally at a concentration of from about 10 mM to about 100 mM.

10. The method of any preceding claim, wherein the sample i) comprises an in vitro transcription reaction.

11. The method of any preceding claim, wherein the sample i) is provided as a solution.

12. The method of claim 11, wherein said contacting the sample with the solid support and the binding solution comprises contacting the solid support with the sample to form a mixture, then contacting the mixture with the binding solution.

13. The method of claim 11 or claim 12, wherein the sample i) is provided at a lower volume than the binding solution ii), optionally wherein the sample i) is provided at a volume of not more than about 60% of the volume of binding solution ii).

14. The method of any preceding claim, wherein the method further comprises:
d) washing the separated solid support with bound RNA with a wash buffer and then separating the solid support with bound RNA from the residual solution,
the wash buffer comprising an aqueous C₁-C₆ alcohol, and/or an aqueous C₂-C₁₀ polyol.

15. The method of claim 14, wherein the wash buffer comprises about 50% v/v to about 90% v/v ethanol, optionally about 60% v/v to about 80% v/v ethanol.

16. The method of claim 14 or claim 15, further comprising repeating step d).

17. The method of any preceding claim, further comprising:
e) contacting the separated solid support with bound RNA with an elution buffer, under conditions that release the bound RNA into the elution buffer, thereby providing an eluate and eluted solid support; and
f) collecting the eluate.

18. The method of claim 17, wherein the elution buffer is or comprises Tris/EDTA, Tris, or water;
optionally wherein the elution buffer comprises Tris/EDTA at pH 8.

19. The method of claim 17 or claim 18, wherein the elution buffer is provided at a volume of less than 100 μL per mg (dry wt) separated solid support with bound RNA.

20. The method of any of claims 17 to 19, wherein the volume of elution buffer is less than the volume of the sample comprising RNA, such that the method both purifies and concentrates the RNA from the sample.

21. The method of claim 20, wherein the volume of the elution buffer is at least about 50% less than the volume of the sample comprising RNA; optionally wherein the volume of the elution buffer is at least about 66% less than the volume of the sample comprising RNA; further optionally wherein the volume of the elution buffer is at least about 75% less than the volume of the sample comprising RNA.

22. The method of any of claims 17 to 21, further comprising:
g) repeating steps a) to f) at least once, wherein the eluted solid support of step f) of the (or each) immediately previous cycle is used as the solid support having a hydrophilic surface in step a) of the following cycle.

23. The method of claim 22, wherein step g) comprises repeating steps a) to f) at least 2, 3, 4, 5 or 6 times.

24. The method of claim 22 or claim 23, wherein the solid support comprises magnetic beads comprising a saturation mass magnetization of about 30 emu/g to about 90 emu/g.

25. The method of any of claims 22 to 24, wherein the eluted solid support of step f) of the (or each) immediately previous cycle is reused as the solid support having a hydrophilic surface in step a) of the following cycle, without any washing of said solid support between each step g) and a).

26. A method for producing a purified ribonucleic acid (RNA) molecule, the method comprising:
a) fixing a first magnetic bead in place by a magnetic field, wherein an in vitro transcription (IVT) template is linked to the first magnetic bead;
b) contacting the first magnetic bead of step a) with a reagent mixture suitable for IVT of the template under condition in which IVT occurs, thereby producing an RNA molecule;
c) separating the RNA molecule from the first magnetic bead, thereby producing the purified RNA molecule;
d) contacting the purified RNA molecule of step c) with a second magnetic bead under conditions that allows for the purified RNA molecule to remain associated with the second magnetic bead during washing;
e) washing of the second magnetic bead while the second magnet bead is fixed in place by a magnetic field;
f) releasing the purified RNA molecule from association with the second magnetic bead, thereby producing a highly purified RNA molecule; and
g) repeating steps a) to f) at least once, wherein the second magnetic bead of step f) of the (or each) immediately previous cycle is reused as the second magnetic bead in step d) of the following cycle, and optionally wherein the first magnetic bead of step c) of the (or each) immediately previous cycle is reused as the first magnetic bead in step a) of the following cycle.

27. The method of claim 26, wherein step g) comprises repeating steps a) to f) at least 2, 3, 4, 5 or 6 times.

28. The method of claim 26 or claim 27, wherein the first magnetic bead is a streptavidin coated magnetic bead and the second bead is a carboxylic acid coated bead.

29. The method of any of claims 26 to 28, wherein the first and/or second magnetic beads comprise a saturation mass magnetization of about 30 emu/g to about 90 emu/g.

30. The method of any of claims 26 to 29, wherein the conditions of contacting step d) comprises contacting the purified RNA molecule with the second magnetic beads in the presence of a binding solution comprising an oligoethylene glycol and a salt, the oligoethylene glycol comprising from about 2 to about 70 ethylene glycol units in linear arrangement, wherein during the contacting the oligoethylene glycol is present at a concentration of at least about 35% v/v and the salt is present at a concentration of between about 1 M and about 2 M.

31. The method of claim 30, wherein the composition of the binding solution is as further defined in any of claims 2 to 9.

32. The method of any of claims 26 to 31, wherein step e) washing comprises use of a wash buffer comprising an aqueous C₁-C₆ alcohol, and/or an aqueous C₂-C₁₀ polyol; optionally wherein the wash buffer comprises about 50% v/v to about 90% v/v ethanol.

33. The method of any of claims 26 to 32, wherein the IVT template is a synthetic DNA fragment or is produced by polymerase chain reaction (PCR);
optionally wherein one or more biotinylated primer is used in the PCR and results in the formation of a biotinylated IVT template.

34. A kit comprising:
a) a binding solution comprising aqueous oligoethylene glycol and a salt, wherein the oligoethylene glycol comprises from about 2 to about 70 ethylene glycol units in linear arrangement, and wherein the oligoethylene glycol is present at a concentration of at least about 35% v/v and the salt is present at a concentration of between about 1 M and about 2 M; and
b) a solid support having a hydrophilic surface.

35. The kit of claim 34, wherein the binding solution is as further defined in any of claims 2 to 9.

36. The kit of claim 34 or 35, further comprising a wash buffer comprising an aqueous C₁-C₆ alcohol and/or an aqueous C₂-C₁₀ polyol,
optionally wherein the wash buffer comprises about 50% v/v to about 90% v/v ethanol,
further optionally wherein the wash buffer comprises about 60% v/v to about 80% v/v ethanol.

37. The kit of any of claims 34 to 36, further comprising an elution buffer;
optionally wherein the elution buffer is or comprises Tris/EDTA, Tris, or water;
further optionally wherein the elution buffer comprises Tris/EDTA at pH 8.

38. Use of a binding solution to separate RNA from a sample solution to a solid support,
wherein the binding solution comprises an oligoethylene glycol at a concentration of at least about 40% v/v and a salt present at a concentration of between about 1.2 M and about 2.5 M,
wherein the oligoethylene glycol comprises from about 2 to about 70 ethylene glycol units in linear arrangement.

39. The use of claim 38, wherein the oligoethylene glycol comprises about 2 to about 20 ethylene glycol units in linear arrangement;
optionally wherein the oligoethylene glycol is or comprises triethylene glycol, tetraethylene glycol, pentaethylene glycol, hexaethylene glycol, or heptaethylene glycol;
further optionally wherein the oligoethylene glycol is or comprises tetraethylene glycol.

40. The use of claim 38 or claim 39, wherein the oligoethylene glycol is present in a concentration of between about 50% v/v and about 70% v/v.

41. The use of any of claims 38 to 40, wherein the salt is selected from or comprises an alkali metal halide, or an alkaline earth metal halide;
optionally wherein the salt is selected from or comprises sodium chloride, potassium chloride, lithium chloride, magnesium chloride, calcium chloride and barium chloride;
further optionally wherein the salt is or comprises sodium chloride.

42. The use of any of claims 38 to 41, wherein the binding solution comprises buffer providing a pH of from about 6 to about 9;
optionally wherein the buffer provides a pH of about 7 to about 8.

43. The use of claim 42, wherein the buffer comprises or is selected from Tris, Tris/EDTA, PBS, citrate, sodium citrate, 2-(N-morpholino)ethanesulfonic acid (MES), or water optionally wherein the buffer is comprises or is selected from Tris, Tris/EDTA, PBS, or water.

44. The use of claim 42 or 43, wherein the buffer comprises Tris, optionally at a concentration of from about 10 mM to about 100 mM.
